SEQUENCE LISTING (3 sequences)

SEQ ID NO: 1 - DNA, 22 bp, Artificial Sequence
  Chemcially Synthesized
gcctcaataaagcttgccttga

SEQ ID NO: 2 - DNA, 18 bp, Artificial Sequence
  Chemcially Synthesized
gggcgccactgctagaga

SEQ ID NO: 3 - DNA, 26 bp, Artificial Sequence
  Chemcially Synthesized
ccagagtcacacaacagacgggcaca

(19) United States
(12) Patent Application Publication
(10) Pub. No.: US 2024/0181051 A1
(43) Pub. Date: Jun. 6, 2024

(54) SIALYDASE LINKED HIV ANTIBODIES AND METHODS OF USE

(71) Applicant: The Wistar Institute of Anatomy and Biology, Philadelphia, PA (US)

(72) Inventor: Mohamed Abdel-Mohsen, Philadelphia, PA (US)

(21) Appl. No.: 18/556,263

(22) PCT Filed: Apr. 19, 2022
(86) PCT No.: PCT/US22/25274

§ 371 Date: Oct. 19, 2023

Related U.S. Application Data
(60) Provisional application No. 63/176,510, filed on Apr. 19, 2021.

(51) Int. Cl. A61K 39/42; A61K 38/47; C07K 16/10; C12N 9/24

(52) U.S. Cl. CPC A61K 39/42; A61K 38/47; C07K 16/1045; C12N 9/2402; C12Y 302/01018; C07K 2319/30

(57) ABSTRACT

Disclosed herein are compositions for targeted desialylation of HIV-infected cells, including neutralizing HIV antibodies conjugated to desialylation enzymes, and methods of use for treating or preventing an HIV infection or a disease or disorder associate with an HIV infection.

### CROSS REFERENCE TO RELATED APPLICATIONS

This application claims priority to U.S. Provisional Application No. 63/176,510, filed Apr. 19, 2021 which is hereby incorporated by reference herein in its entirety.

### STATEMENT REGARDING FEDERALLY SPONSORED RESEARCH OR DEVELOPMENT

This invention was made with Government support under AI165079-02 awarded by the National Institutes of Health. The Government has certain rights in the invention.

### TECHNICAL FIELD

The present invention relates to compositions comprising HIV antibodies linked to a domain for desialydation of HIV infected cells, and functional fragments thereof, in vivo, and methods of use thereof for preventing and/or treating HIV infection or a disease or disorder associated therewith in a subject by administering said compositions.

### BACKGROUND

The barrier to HIV eradication is the ability of the virus to establish persistent infection in CD4⁺ T cells and possibly other cell types (Chun et al., 1995, Nature medicine 1, 1284-1290; Wong et al., 1997, Science 278, 1291-1295; Finzi et al., 1997, Science 278, 1295-1300; Chun et al., 1997, Nature 387, 183-188; Chun et al., 1997, Proc Natl Acad Sci USA 94, 13193-13197; Yukl et al., 2013, J Infect Dis 208, 1212-1220; Estes et al., 2017, Nat Med 23, 1271-1276). A “functional HIV cure” may be established by enabling antiretroviral therapy (ART)-independent suppression of HIV (Saez-Cirion et al., 2013, PLoS pathogens 9, e1003211). One proposed approach to reach this goal is “shock and kill” (Siliciano et al., 2013, Current opinion in HIV and AIDS 8, 318-325). In this approach, latency reversal agents (LRAs) are administered to reverse HIV latency and induce viral production; however, reversing latency is only the first step (shock). The second step (kill) requires efficient immune responses to clear reactivated cells. Clinical trials involving LRAs have shown that immune responses of HIV-infected ART-treated individuals cannot clear reactivated reservoirs, suggesting that adjuvant immunotherapy is needed (Elliott et al., 2014, PLoS Pathog 10, e1004473; Olesen et al., 2015, J Virol. 89, 10176-89; Spivak et al., 2014, Clin Infect Dis 58, 883-890; Archin et al., 2014, J Infect Dis 210, 728-735; Archin et al., 2012, Nature 487, 482-485; Eliott et al., 2015, The lancet. HIV 2, e520-529; Sogaard et al., 2015, PLoS Pathog 11, e1005142). One potential adjuvant strategy is to enhance the cytotoxicity of natural killer (NK) cells during viral reactivation (achieved by LRAs or by ART-cessation). Developing a strategy to achieve this goal would require a better understanding of the factors that determine NK functions against HIV-infected cells.

The functions of NK cells can be influenced by the cell-surface glycosylation of their target cells. NK cells express several cell-surface lectins (glycan-binding proteins), including two belonging to the Siglec family: Siglec-7 and Siglec-9. Siglecs (Sialic acid-binding immunoglobulin-type lectins) are ITIM-containing, MHC-independent inhibitory receptors that control host immune responses by interacting with Sialic-acid containing glycans on the surface of target cells. Siglec-7 is expressed on almost all NK cells and binds to α2-8 Sialic acid. Decreased levels of Siglec-7 have been described as a marker for dysfunctional NK cells in HIV viremic individuals (Brunetta et al., 2009, Blood 114, 3822-3830; Varchetta et al., 2013, Retrovirology 10, 154; Zulu et al., 2017, AIDS Res Hum Retroviruses 33, 1205-1213). Quite differently, Siglec-9 is selectively expressed on a subset of the CD56^{dim} NK cells (the cytolytic subset of NK cells) (Belisle et al., 2010, Mol Cancer 9, 118) and binds to α2-3 Sialic acid. The binding of Siglec-9 to α2-3 Sialic acid on target cells induces an inhibitory signal transduction cascade by recruiting the tyrosine phosphatase SHP-1, which counteracts the phosphorylation-mediated activation of other signaling molecules (Avril et al., 2004, Journal of immunology 173, 6841-6849; Crocker et al., 2007, Nature reviews. Immunology 7, 255-266). As such, Siglec-9 functions as a glyco-immune negative checkpoint, analogous to the PD1 checkpoint on activated CD8⁺ T cells.

Siglec-9 continues to transmit inhibitory signals into NK cells even when target cells have lost the expression of MHC class I molecules (missing-self) or when the classical inhibitory NK receptors are inefficiently engaged (Daly et al., 2019, Frontiers in immunology 10, 1047). This MHC independence of the Siglec-9 molecule makes it perfect for exploitation by cancer cells or virally-infected cells to evade host immune surveillance. Indeed, emerging evidence suggests that the Siglec-9⁺ CD56^{dim} NK population plays an important role in regulating NK cytotoxicity against cancer cells and hepatitis B virus (HBV)-infected cells (Jandus et al., 2014, The Journal of clinical investigation 124, 1810-1820; Zhao et al., 2018, Frontiers in immunology 9, 1124). Despite this, the role of Siglec-9 expression on NK cells during HIV infection has not been elucidated.

Thus there is need in the art for therapeutics that enhance the ability of NK cells to target antigen-producing HIV-infected cells. The current invention satisfies this need.

### SUMMARY

In one embodiment, the invention relates to a composition comprising a targeting domain specific for binding to a human immunodeficiency virus (HIV) antigen and a domain for desialylation of HIV-infected cells.

In one embodiment, the domain for desialylation of HIV-infected cells comprises a neuraminidase enzyme, or a fragment or variant thereof. In one embodiment, the neuraminidase enzyme is NEU1, NEU2, NEU3, NEU4, or a bacterial or viral neuraminidase.

In one embodiment, the composition comprises an anti-HIV antibody conjugated to a domain for desialylation of HIV-infected cells. In one embodiment, the anti-HIV antibody comprises PGDM1400, PGT121, or a combination thereof.

In one embodiment, the domain for desialylation of HIV-infected cells comprises a neuraminidase enzyme, or a fragment or variant thereof. In one embodiment, the neuraminidase enzyme is NEU1, NEU2, NEU3, NEU4, or a bacterial or viral neuraminidase.

In one embodiment, the composition comprises a pharmaceutically acceptable excipient.

In one embodiment, the invention relates to a method of preventing or treating HIV or a disease or disorder associated with HIV infection in a subject, the method comprising administering to the subject a composition comprising a targeting domain specific for binding to a human immunodeficiency virus (HIV) antigen and a domain for desialylation of HIV-infected cells.

In one embodiment, the domain for desialylation of HIV-infected cells comprises a neuraminidase enzyme, or a fragment or variant thereof. In one embodiment, the neuraminidase enzyme is NEU1, NEU2, NEU3, NEU4, or a bacterial or viral neuraminidase. In one embodiment, the composition comprises an anti-HIV antibody conjugated to a domain for desialylation of HIV-infected cells.

In one embodiment, the anti-HIV antibody comprises PGDM1400, PGT121, or a combination thereof. In one embodiment, the domain for desialylation of HIV-infected cells comprises a neuraminidase enzyme, or a fragment or variant thereof.

In one embodiment, the neuraminidase enzyme is NEU1, NEU2, NEU3, NEU4, or a bacterial or viral neuraminidase.

In one embodiment, the composition comprises a pharmaceutically acceptable excipient.

In one embodiment, the disease is acquired immunodeficiency syndrome (AIDS).

In one embodiment, the invention relates to a nucleic acid molecule encoding a fusion molecule comprising targeting domain specific for binding to a human immunodeficiency virus (HIV) antigen and a domain for desialylation of HIV-infected cells. In one embodiment, the domain for desialylation of HIV-infected cells comprises a neuraminidase enzyme, or a fragment or variant thereof. In one embodiment, the neuraminidase enzyme is selected from the group consisting of NEU1, NEU2, NEU3, NEU4, or a bacterial or viral neuraminidase.

In one embodiment, the nucleic acid molecule encodes an anti-HIV antibody conjugated to a domain for desialylation of HIV-infected cells. In one embodiment, the nucleic acid molecule encodes PGDM1400 or PGT121.

In one embodiment, the invention relates to a fusion molecule comprising targeting domain specific for binding to a human immunodeficiency virus (HIV) antigen and a domain for desialylation of HIV-infected cells. In one embodiment, the domain for desialylation of HIV-infected cells comprises a neuraminidase enzyme, or a fragment or variant thereof. In one embodiment, the enzyme is selected from the group consisting of NEU1, NEU2, NEU3, NEU4, or a bacterial or viral neuraminidase. In one embodiment, the fusion molecule comprises an anti-HIV antibody conjugated to a domain for desialylation of HIV-infected cells.

### BRIEF DESCRIPTION OF THE DRAWINGS

FIG. 1 depicts the gating strategy used in the experiments. A representative example of the gating strategy used for the phenotyping of CD56^{dim} NK cells. First, the most stable time of acquisition was selected. Single lymphocytes were then gated and characterized as negative for aqua-blue viability dye, CD14, CD19 (in the dump gate), and CD3. Any remaining monocyte populations were then excluded by selecting HLA-DR⁻ cells. Total NK cells were selected based on their expression of CD56 (magenta); CD56^{dim} NK cells were gated separately (blue) for further analyses.

FIG. 2A through FIG. 2D depict the expression of Siglec-9⁺ CD56^{dim} NK cells during HIV infection. FIG. 2A depicts an overlay plots showing the distribution of Siglec-9⁺ CD56^{dim} NK cells (red) compared with total NK cells (blue) and non-T cell lymphocytes (grey). FIG. 2B through FIG. 2C depict representative plots showing the frequency (FIG. 2B) and expression (MFI overlay) (FIG. 2C) of Siglec-9 in total CD56^{dim} NK cells in HIV− (blue line), ART+ HIV+ (orange line), and viremic HIV+ (red line) individuals. FIG. 2D depicts the decreased frequency of Siglec-9⁺ CD56^{dim} NK cells during HIV infection compared to HIV− controls. Lines in graphs indicate the median of the group. ** p<0.01. Mann-Whitney rank test was used to compare between groups.

FIG. 3A through FIG. 3C depict the phenotype of Siglec-9⁺ CD56^{dim} NK cells. FIG. 3A depicts the global t-SNE visualization of Siglec-9⁺ CD56^{dim} NK cells for all individuals pooled, with Siglec-9⁺ CD56^{dim} NK cells from HIV−, HIV+ ART, and HIV+ viremic individuals concatenated and overlayed (dimensionality reduction performed from 234,000 cells in 21 dimensions, 10,000 iterations, excluding parameters used to define the population: time, FSC, SSC, viability, CD14, CD19, CD3, CD8, and Siglec-9). Bottom: t-SNE projections of the 18 indicated proteins expression. FIG. 3B depicts heatmaps showing the percentages of Siglec-9⁺ and Siglec-9⁻ CD56^{dim} NK cells expressing the indicated activation and inhibitory markers in HIV−, ART HIV+ and viremic HIV+ individuals. FIG. 3C depicts a comparative analyses of frequency (% positive) and expression (MFI of the positive population) of Siglec-7, CD16, CD38 CD161, NKp30, KIR3DL1, NKG2A, TIGIT, Perform, and DNAM-1 on Siglec-9⁺ vs. Siglec-9⁻ CD56^{dim} NK cells. Left: Representative flow plots and histograms from an HIV+ ART donors are shown. Numbers inside the plots represent the gated percentage within the parent population. Mann-Whitney rank test was used to compare between groups. Paired Wilcoxson test was used to compare Siglec-9⁺ and Siglec-9⁻ within each group. ***p<0.001, ** p<0.01, *p<0.05.

FIG. 4A through FIG. 4D depict the phenotypic characterization of Siglec-9⁺ CD56^{dim} NK subpopulation and association with levels of CD4⁺ T cell-associated HIV DNA. FIG. 4A depicts t-SNE visualization of 8 Siglec-9⁺ CD56^{dim} NK cell clusters identified by FlowSOM clustering. FIG. 4B depicts a heatmap and hierarchical clustering of MFI of each marker within the indicated FlowSOM clusters. FIG. 4C depicts the percentage of Siglec-9⁺ CD56^{dim} NK cells from each group in each FlowSOM cluster. Bars indicate the median and interquartile range of the group. Each symbol represents an individual: HIV− in blue, ART HIV+ in orange, viremic HIV+ in red. ** p<0.01, *p<0.05. Mann-Whitney rank test was used to compare between groups. FIG. 4D depicts the spearman correlation between the frequency of Siglec-9⁺ CD56^{dim} NK cells and cell-associated HIV DNA copies per million CD4⁺ T cells during ART-suppressed HIV infection.

FIG. 5A through FIG. 5F depict data demonstrating that the Siglec-9⁺ CD56^{dim} NK cells exhibit higher cytotoxicity towards HIV+ cells compared to Siglec-9⁻ CD56^{dim} NK cells, but this cytotoxicity is being restrained by the inhibitory nature of the Siglec-9 molecule. FIG. 5A depicts a representative example of depletion of Siglec-9⁺ NK cells. FIG. 5B depicts data demonstrating that Siglec-9^{depleted} NK cells exhibit lower cytotoxicity towards HIV-infected HUT78/SF2 targets compared to total NK cells. Cytotoxicity was assessed using NK degranulation, LDH release, and CFSE/SYTOX Red assay. NK degranulation measured as CD107⁺ IFNγ+. E:T=Effector to target ratio. FIG. 5C depicts FACS sorted Siglec-9⁺ CD56^{dim} NK cells exhibit higher cytotoxicity towards HIV+ CEM.NKR targets compared to Siglec-9⁻ CD56^{dim} NK cells. Cytotoxicity was assessed using LDH release assay (E:T=10:1) (left panel). Analysis of NK degranulation (E:T=4:1) was made on total NK cells gated on Siglec-9⁺ or Siglec-9⁻ CD56^{dim} NK cell subsets (right two panels). FIG. 5D depicts data demonstrating that blocking Siglec-9 enhances NK killing capacity. HIV-infected HUT78/SF2 cells were used as target cells, and total or siglec9^{depleted} NK cells from HIV− donors were used as effector cells in the presence/absence of isotype control or Siglec-9 blocking Ab. Cytotoxicity was assessed by LDH release assay (E:T=20:1). FIG. 5E and FIG. 5F depict data demonstrating that blocking Siglec-9 enhanced the ability of Siglec-9⁺ CD56^{dim} NK cells to target HIV-infected CEM.NKR cells. Cytotoxicity was assessed by (FIG. 5E) the LDH release assay (E:T=10:1) or (FIG. 5F) NK degranulation (E:T=4:1). Analysis of NK degranulation (E:T=4:1) was made on total NK cells gated on Siglec-9⁺ or Siglec-9⁻ CD56^{dim} NK cell subsets. All assays from each donor were done in multiple replicates (2-4), and the average of these replicates was used for analysis. Statistical analysis was performed using paired t-tests.

FIG. 6A through FIG. 6C depict data demonstrating infection of HUT78 and CEM.NKR cells with HIV. FIG. 6A depicts a representative example of HUT78 cells infection with HIV SF2. Cells were analyzed for intracellular p24 by staining with anti-p24 RD1 antibody. FIG. 6B depicts cell surface Siglec-9 ligand expression. Equal number of indicated cells were incubated with varying amounts of recombinant human Siglec-9 Fc protein. Binding of Siglec-9 Fc to cells was revealed using PE anti-human Fc fluorescent secondary antibody. FIG. 6C depicts a representative example of CEM.NKR cells infection with HIV DH12. Cells incubated for 72 h on RetroNectin precoated dishes with immobilized HIV were analyzed for intracellular p24 by staining with anti-p24 RD1.

FIG. 7A and FIG. 7B depict data demonstrating the Siglec-9⁺ CD56^{dim} cells exhibit lower cytotoxicity towards K562 cancer cells compared to Siglec-9⁻ CD56^{dim} Cytotoxicity was assessed using (FIG. 7A) NK degranulation and (FIG. 7B) IFNγ production (E:T=4:1). Total NK cells were gated on Siglec-9⁺ or Siglec-9⁻ CD56^{dim} NK cell subsets. Assays from each donor were done in multiple replicates (3), and the average of these replicates was used for analysis. Statistical analysis was performed using paired t-tests.

FIG. 8 depicts data demonstrating that produced bNAbs bind to HIV-infected cells. Representative examples of staining HUT78 HIV-negative and HUT78/SF2 HIV+ cells with 3BNC117, PGT151, and NIH45-46. PE-fluorescent secondary antibody was used for detection using flow cytometry.

FIG. 9A through FIG. 9E depict data demonstrating bNAb-Sialidase conjugates selectively target HIV+ cells for desialylation. FIG. 9A depicts preparation of site-specifically labeled HIV bNAb-Sialidase (bNAb-STSia) conjugates. Antibody-binding peptide (light blue) genetically fused with Sialidase (yellow) is conjugated to bNAb using pClick. pClick enables a site-specific conjugation between the antibody-binding peptide with payload and Lys337 of antibodies (top); PAGE-SDS with non-reducing buffer of bNAb-Sia conjugates (bottom). FIG. 9B and FIG. 9C depict data demonstrating that a mixture of HUT78 cells (HIV^{-negative}) and HUT78/SF2 cells (HIV⁺) was treated with escalating doses of NIH45-46-STSia. HIV gp120 was measured by a secondary antibody to NIH45-46, and Sialic acid levels were measured as binding to SNA lectin. Representative flow plots (FIG. 9B). The fold reduction shows that sialic acid was reduced by >7 fold on HIV⁺ cells compared to HIV^{-negative} cells (FIG. 9C). FIG. 9D and FIG. 9E depict data demonstrating that cells were treated as in B/C but using the 3BNC117-STSia conjugate (FIG. 9D) or the PGT151-STSia conjugate (FIG. 9E). STSia=Sialidase from Salmonella typhimurium.

FIG. 10A through FIG. 10C depict data demonstrating desialylation of HIV-infected target cells potentiates NK cytotoxicity. FIG. 10A depicts preparation p24 analysis of HIV HXB2-infected CEM.NKR CCR5+ Luc+ cells. Cells incubated for 72 h on RetroNectin precoated dishes with immobilized HIV were analyzed for intracellular p24 by staining with anti-p24 RD1. FIG. 10B depicts CEM.NKR CCR5+ Luc+ cells treated with 200 nM STSia for 1 h at 37° C. were incubated with 1 μg recombinant human Siglec-9 Fc protein. Binding of Siglec-9 Fc to cells was revealed using PE anti-human Fc fluorescent secondary antibody. FIG. 10C depicts HIV-infected CEM-NKR CCR5⁺ Luc⁺ cells were treated with indicated amounts of STSia or bNAbs. Treated cells were then cocultured with effector NK cells (E:T 10:1). Luminescence was measured as a marker of intact (unkilled) HIV+ cells. Statistical analysis was performed using one-way ANOVA from four technical replicates.

FIG. 11A through FIG. 11F depict data demonstrating bNAbs-STSia conjugates promote higher NK cytotoxicity against HIV+ cells compared to bNAbs alone. FIG. 11A through FIG. 11C depict a killing assay using HIV-infected CEM-NKR CCR5⁺ Luc⁺ cells as targets and HIV-negative primary NK cells as effectors (n=3 donors). Luminescence was measured as a marker of intact (unkilled) HIV+ cells. FIG. 11A depicts NIH45-46 and its conjugate. FIG. 11B depicts 3BNC117 and its conjugate. FIG. 11C depicts PGT151 and its conjugate. P values were calculated using one-way ANOVA comparing all conditions against the HIV+ cells alone. FIG. 11D through FIG. 11F depict a killing assay using HIV-infected CEM-NKR CCR5⁺ Luc⁺ cells as targets and HIV-negative primary NK cells as effectors (n=4 donors). Cytotoxicity was assessed by the LDH release assay. (FIG. 11D) NIH45-46 and its conjugate. (FIG. 11E) 3BNC117 and its conjugate. (FIG. 11F) PGT151 and its conjugate. P values were calculated using paired T-tests.

FIG. 12 depicts a model of how HIV bNAb-Sialidase conjugates may increase the cytotoxicity of Siglec-9⁺ NK cells against HIV-infected cells. Left two panels: The Siglec-9⁺ CD56^{dim} NK subset has high cytolytic activity, possibly due to elevated expression of several NK activating receptors and reduced expression of the inhibitory NKG2A, compared to Siglec-9⁻ CD56^{dim} NK cells. However, Siglec-9 itself is an inhibitory receptor whose signaling restrains the cytolytic ability of these otherwise highly cytotoxic Siglec-9⁺ CD56^{dim} NK cells by binding to Sialic acid attached to protein or lipid backbones on the surface of target cells. Right panel: Siglec/Sialic acid interactions are being pursued as an approach to enhance NK cell cytotoxicity against cancer using antibodies conjugated to Sialidase. Sialidase conjugated to HIV bNAbs were developed that could be used in conjunction with strategies that reactivate HIV latently-infected cells to enhance NK cells' capacity to clear HIV+ cells.

FIG. 13 depicts clinical data of the study participants whose cells were used for the experiments in FIGS. 2, 3, 4A-C.

FIG. 14 depicts clinical data of the study participants whose cells were used for the experiments in FIG. 4D.

### DETAILED DESCRIPTION

The present invention relates to compositions and methods for targeted desialylation of HIV infected cells. In some embodiments, the invention comprises HIV antigen binding molecules conjugated to a desialylation domain, such as a molecule comprising neuraminidase activity. In some embodiments, the molecule comprising neuraminidase activity is NEU1, NEU2, NEU3, NEU4, or a bacterial or viral neuraminidase.

In some embodiments, the HIV binding molecule comprises an anti-HIV antibody. In some embodiments, the anti-HIV antibody is a neutralizing antibody. In some embodiments, the invention provides methods of treating an HIV infection, or a disease or disorder associated therewith, using the compositions described herein.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“About” as used herein when referring to a measurable value such as an amount, a temporal duration, and the like, is meant to encompass variations of 20%, +10%, +5%, +1%, ±0.1%, less than ±0.1%, or any percentage therebetween from the specified value, as such variations are appropriate to perform the disclosed methods.

There term “in combination with” is used herein to that two or more indicated events occurred concurrently or that a first event occurs sequentially with one or more additional event.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

The term “fusion protein” used herein refers to two or more peptides, polypeptides, or proteins operably linked to each other.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

Unless otherwise specified, a “nucleotide sequence encoding an amino acid sequence” includes all nucleotide sequences that are degenerate versions of each other and that encode the same amino acid sequence.

“Immune response” as used herein may mean the activation of a host's immune system, e.g., that of a mammal, in response to the introduction of one or more nucleic acids and/or peptides. The immune response can be in the form of a cellular or humoral response, or both.

A “label” or a “detectable moiety” is a composition detectable by spectroscopic, photochemical, biochemical, immunochemical, chemical, magnetic resonance imaging, or other physical means. For example, useful detectable moieties include 32P, fluorescent dyes, electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, standard superparamagnetic iron oxide (“SSPIO”), SSPIO nanoparticle aggregates, polydisperse superparamagnetic iron oxide (“PSPIO”), PSPIO nanoparticle aggregates, monochrystalline SPIO, monochrystalline SPIO aggregates, monochrystalline iron oxide nanoparticles, monochrystalline iron oxide, other nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Detectable moieties also include any of the above compositions encapsulated in nanoparticles, particles, aggregates, coated with additional compositions, derivatized for binding to a targeting agent (e.g. antibody or antigen binding fragment). Any method known in the art for conjugating an antibody to the label may be employed, e.g., using methods described in Hermanson, Bioconjugate Techniques 1996, Academic Press, Inc., San Diego.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

Nucleic acids may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleotide sequence is “operably linked” when it is placed into a functional relationship with another nucleotide sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The term “diagnostically acceptable” is used synonymously with “physiologically acceptable” and “pharmacologically acceptable” and refers to diagnostic compositions.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative. In some embodiments, conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Following expression, proteins can be isolated. The term “purified” or “isolated” as applied to any of the proteins described herein (e.g., a conjugate, antibody or antigen-binding fragment thereof described herein) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of a disease or disorder is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

For the recitation of numeric ranges herein, each intervening number there between with the same degree of precision is explicitly contemplated.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. An additional examples, for the range of 6-9, the numbers 7 and 8 are contemplated in addition to 6 and 9, and for the range 6-7, the number 6.0, 6.1, 6.2, 6.3, 6.4, 6.5, 6.6, 6.7, 6.8, 6.9, and 7.0 are explicitly contemplated. This applies regardless of the breadth of the range.

### Compositions

Siglec-9 is an MHC-independent inhibitory receptor expressed on a subset of natural killer (NK) cells. Siglec-9 restrains NK cytotoxicity by binding to sialoglycans on target cells. IN various embodiments, the invention provides compositions and methods to selectively disrupt Siglec/sialoglycan interactions between NK and HIV-infected cells by conjugating Sialidase to HIV-targeted therapeutic molecules. These conjugates selectively desialylated HIV-infected cells and enhanced NK capacity to kill them.

In some embodiments, the invention provides compostions comprising an HIV targeting domain conjugated to a domain for desialylation of HIV-infected cells. In some embodiments, the molecule for desialylation of HIV-infected cells comprises a neuraminidase (or sialidase) enzyme, or a variant thereof. Exemplary neuraminidases include, but are not limited to, NEU1, NEU2, NEU3, NEU4, or a bacterial or viral neuraminidase (e.g., influenza neuraminidase).

In various embodiments of the invention, the desialylation domain is conjugated directly a molecule comprising an HIV targeting domain (e.g., a neutralizing anti-HIV antibody). In some embodiments, the invention relates to a composition comprising a delivery vehicle comprising a desialylation domain and further comprising an HIV targeting domain wherein each of the desialylation domain and HIV targeting domain are conjugated to components of the delivery vehicle. Exemplary delivery vehicles that can be conjugated to an HIV targeting and desialylation domain of the invention include, but are not limited to, liposomes, microparticles, nanoparticles, lipid nanoparticles, and protein particles. In some embodiments, the delivery vehicle further comprises or encapsulates a therapeutic agent for the treatment of an HIV infection.

### Conjugation

Exemplary methods of conjugation of a desialylation domain to an HIV targeting molecule or a delivery vehicle can include, but are not limited to, covalent bonds, electrostatic interactions, and hydrophobic (“van der Waals”) interactions. In one embodiment, the conjugation is a reversible conjugation, such that the HIV targeting molecule or a delivery vehicle can be disassociated from the desialylation domain upon exposure to certain conditions or chemical agents. In another embodiment, the conjugation is an irreversible conjugation, such that under normal conditions the HIV targeting molecule or a delivery vehicle does not dissociate from the desialylation domain.

In some embodiments, the HIV targeting molecule or a delivery vehicle and the desialylation domain, are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### Targeting Domain

In one embodiment, the composition comprises a targeting domain that directs the sialidase conjugated therapeutic molecule of the invention to an HIV-infected cell. The targeting domain may comprise a nucleic acid, peptide, antibody, small molecule, organic molecule, inorganic molecule, glycan, sugar, hormone, and the like that targets the sialidase conjugated therapeutic molecule to an HIV-infected cell. In certain embodiments, the sialidase conjugated therapeutic molecule comprises multivalent targeting, wherein the particle comprises multiple targeting mechanisms described herein. In certain embodiments, the targeting domain may be chosen to recognize a ligand that acts as a cell surface marker on HIV-infected cells. Such a target can be a protein, protein fragment, antigen, or other biomolecule that is associated with HIV infection. In some embodiments, the targeting domain is an affinity ligand which specifically binds to an HIV antigen. In some embodiments, the targeting domain may be a therapeutic molecule (e.g., a neutralizing antibody). In some embodiments, the targeting domain may be covalently attached to a delivery vehicle comprising a therapeutic molecule, such as through a chemical reaction between the targeting domain and the delivery vehicle. In some embodiments, the targeting domain is an additive in a delivery vehicle. Targeting domains of the instant invention include, but are not limited to, antibodies, antibody fragments, proteins, peptides, and nucleic acids.

### Peptides

In one embodiment, the targeting domain of the invention comprises a peptide. In certain embodiments, the peptide targeting domain specifically binds to a target of interest (e.g., an HIV antigen).

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In one embodiment, the targeting domain of the invention comprises an isolated nucleic acid, including for example a DNA oligonucleotide and a RNA oligonucleotide. In certain embodiments, the nucleic acid targeting domain specifically binds to an HIV antigen. For example, in one embodiment, the targeting domain comprises a nucleotide sequence that specifically binds to an HIV antigen.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

### Antibodies

In one embodiment, the targeting domain of the invention comprises an antibody, or antibody fragment. In certain embodiments, the antibody targeting domain specifically binds to an HIV antigen. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Recombinant Nucleic Acid Sequence

In some embodiments, the composition can comprise a recombinant nucleic acid sequence encoding a fusion molecule comprising sialidase linked to an HIV targeting domain. The recombinant nucleic acid sequence can encode a fusion molecule comprising sialidase linked to a synthetic antibody (e.g., and HIV antibody or ScFv antibody fragment), a fragment thereof, a variant thereof, or a combination thereof.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Heavy Chain Polypeptide

The recombinant nucleic acid sequence construct can include a heterologous nucleic acid encoding a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The recombinant nucleic acid sequence construct can include a heterologous nucleic acid sequence encoding a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Protease Cleavage Site

The recombinant nucleic acid sequence construct can include a heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides. In one embodiment, the linker sequence is a G4S linker sequence.

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vector

The one or more vectors may be circular plasmid, which may transform a target cell by integration into the cellular genome or exist extrachromosomally (e.g., autonomous replicating plasmid with an origin of replication). The vector can be an expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Antibody

In some embodiments, the invention relates to a molecule comprising sialidase conjugated to an anti-HIV antibody a fragment thereof, a variant thereof, or a combination thereof. The antibody can bind or react with an HIV antigen. In some embodiments, the antibody is a neutralizing antibody, a fragment thereof, or a variant thereof. In some embodiments, the antibody is a DNA encoded monoclonal antibody, a fragment thereof, or a variant thereof. In some embodiments the fragment is an ScFv fragment. In some embodiments, the antibody is a bispecific antibody, a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific or bifunctional antibody as described below in more detail.

As described above, the antibody can be generated in the subject upon administration of a nucleic acid molecule encoding the antibody to the subject. The antibody may have a half-life within the subject. In some embodiments, the antibody may be modified to extend or shorten its half-life within the subject. Such modifications are described below in more detail.

### ScFv Antibody

In one embodiment, the targeting domain of the sialidase fusion molecule of the invention is a ScFv antibody fragment. In one embodiment, ScFv relates to a Fab fragment without the of CH1 and CL regions. Thus, in one embodiment, the ScFv relates to a Fab fragment comprising the VH and VL. In one embodiment, the ScFv comprises a linker between VH and VL.

### Monoclonal Antibodies

In one embodiment, the targeting domain of the sialidase fusion molecule of the invention comprises an anti-HIV antibody. The antibodies may be intact monoclonal antibodies, and immunologically active fragments (e.g., a Fab or (Fab)₂ fragment), a monoclonal antibody heavy chain, or a monoclonal antibody light chain.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

### Bispecific Antibody

In one embodiment, the targeting domain of the sialidase fusion molecule of the invention a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites is capable of an tumor antigen. In some embodiments, the binding site included in the Fab fragment is a binding site specific for a tumor antigen. In some embodiments, the binding site included in the single chain Fv fragment is a binding site specific for an HIV antigen.

In some embodiments, one of the binding sites of a bispecific antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments, it encompasses .gamma.-chains and .delta.-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds an HIV antigen and the second binding site binds a T cell specific receptor molecule and/or a natural killer (NK) cell specific receptor molecule.

In some embodiments, the first binding site of the antibody molecule binds an HIV antigen, and the second binding site binds CD3, TCR, CD28, CD16, NKG2D, Ox40, 4-1BB, CD2, CD5, CD40, FcgRs, FceRs, FcaRs or CD95.

### Bifunctional Antibody

In some embodiments, the invention provides bifunctional antibodies. The bifunctional antibody can bind or react with the HIV antigen, and is also modified by being conjugated to sialidase to impart an additional functionality to the antibody beyond recognition of and binding to the HIV antigen.

### Antigen

In one embodiment, the anti-HIV antibody (e.g., bNAb, ScFv antibody fragment, bispecific antibody, bifunctional antibody) is directed to an HIV antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

### Human Immunodeficiency Virus (HIV) Antigen

The HIV antigen can be a subtype A envelope protein, subtype B envelope protein, subtype C envelope protein, subtype D envelope protein, subtype B Nef-Rev protein, Gag subtype A, B, C, or D protein, MPol protein, a nucleic acid or amino acid sequences of Env A, Env B, Env C, Env D, B Nef-Rev, Gag, or any combination thereof.

In one embodiment, the synthetic antibody specific for HIV is a synthetic antibody, or a combination thereof, encoded by a nucleic acid molecule as described in International Patent Publication No. WO 2018/183294, which is incorporated herein by reference in its entirety. In one embodiment, the synthetic antibody specific for HIV is PGDM1400 or PGT121.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Polypeptide Therapeutic Agents

In other related aspects, the therapeutic agent includes an isolated peptide that modulates a target. For example, in one embodiment, the peptide of the invention inhibits or activates a target directly by binding to the target thereby modulating the normal functional activity of the target. In one embodiment, the peptide of the invention modulates the target by competing with endogenous proteins. In one embodiment, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Antibody Therapeutic Agents

The invention also contemplates a delivery vehicle comprising an antibody, or antibody fragment, specific for a target. That is, the antibody can inhibit a target to provide a beneficial effect.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Therapeutic Methods

In some embodiments, the invention provides methods of treatment of a disease or disorder associated with HIV infection in a subject comprising delivering a sialidase conjugated, HIV targeted, therapeutic agent of the invention. In some embodiments, the disease or disorder associated with HIV infection is AIDS.

In some embodiments, the invention provides methods for targeted delivery of a composition the treatment of HIV or a disease or disorder associated with HIV infection, wherein the composition comprises a desialylation domain and further comprises a moiety for targeting an HIV antigen. In some embodiments, the composition comprises an anti-HIV antibody conjugated to a desialylation domain. In some embodiments, the desialylation domain comprises a neuraminidase enzyme.

Also provided herein is a method of treating, protecting against, and/or preventing a disease or disorder associated with HIV infection in a subject in need thereof by administering a composition comprising a desialylation domain and further comprising a moiety for targeting an HIV antigen. In some embodiments, the composition comprises an anti-HIV antibody conjugated to a desialylation domain. In some embodiments, the desialylation domain comprises a neuraminidase enzyme.

The method can include administering the composition to the subject. Administration of the composition to the subject can be done using any appropriate method of delivery, including but not limited to, those described above.

In one embodiment, the composition comprises a neutralizing antibody specific for binding to an HIV antigen. Upon administration of the antibody to the subject, the antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 pg to 10 mg active component/kg body weight/time, and can be 20 pg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Thus, various modifications of the invention in addition to those shown and described herein will be apparent to those skilled in the art from the foregoing description. Such modifications are also intended to fall within the scope of the appended claims.

### Example 1: Siglec-9 Defines and Restrains a Natural Killer Subpopulation Highly Cytotoxic to HIV-Infected Cells

In this study, a Siglec-9⁺ CD56^{dim} NK subpopulation is identified as a highly cytotoxic NK subpopulation against HIV⁺ cells. The cytotoxicity of this subpopulation is restrained by the inhibitory nature of the Siglec-9 molecule itself. Harnessing the cytotoxic capacity of Siglec-9⁺ CD56^{dim} NK subpopulation, which is dampened by Siglec-9 expression, is a novel approach to control HIV infection during and/or after ART. Towards this goal, a method to selectively disrupt the Siglec/sialoglycan interactions between NK cells and HIV-infected cells was developed. This approach showed specificity and efficacy in enhancing NK activity against HIV-infected cells in vitro.

The cytotoxic potential of NK cells is regulated through the balance of opposing signals delivered by inhibitory and activating cell surface receptors (Lanier et al., 2005, Annu Rev Immunol 23, 225-274; Cerwenka et al., 2001, Nature reviews. Immunology 1, 41-49; Wu et al., 2003, Adv Cancer Res 90, 127-156). HIV infection induces phenotypic changes in NK cells and reduces their cytotoxicity (Scully et al., 2016, Curr HIV/AIDS Rep 13, 85-94), and some of these changes persist even after ART (Mavilio et al., 2005, Proceedings of the National Academy of Sciences of the United States of America 102, 2886-2891). Among the emerging inhibitory receptors on NK cells are the Siglecs (Belisle et al., 2010, Mol Cancer 9, 118; Daly et al., 2019, Frontiers in immunology 10, 1047; Jandus et al., 2014, The Journal of clinical investigation 124, 1810-1820; Zhao et al., 2018, Frontiers in immunology 9, 1124; Stanczak et al., 2018, The Journal of clinical investigation 128, 4912-4923; Zhang et al., 2000, J Biol Chem 275, 22121-22126). Whereas the role of Siglec-7 in HIV pathogenies has been studied (Brunetta et al., 2009, Blood 114, 3822-3830; Varchetta et al., 2013, Retrovirology 10, 154; Zulu et al., 2017, AIDS Res Hum Retroviruses 33, 1205-1213), the role of Siglec-9⁺ CD56^{dim} NK cells in HIV infection has never been elucidated despite the growing appreciation of their role as glyco-immune negative checkpoints in cancer and HBV infection.

In both cancer and HBV pathology, the frequency of cytotoxic Siglec-9⁺ NK cell population is reduced (Jandus et al., 2014, The Journal of clinical investigation 124, 1810-1820; Zhao et al., 2018, Frontiers in immunology 9, 1124; Xiao et al., 2016, Proc Natl Acad Sci USA 113, 10304-10309; Hudak et al., 2014, Nature chemical biology 10, 69-75). During HBV infection, the percentage of Siglec-9⁺ NK cells inversely correlates with HBV DNA (Zhao et al., 2018, Frontiers in immunology 9, 1124). In this study it was found that HIV infection reduced the levels of Siglec-9⁺ CD56^{dim} NK cells. It was confirmed that these cells exhibit an activated phenotype with higher frequencies of activating receptors and markers (NKp30, CD38, CD16, DNAM-1, perforin) and lower expression of the inhibitory receptor NKG2A, compared to Siglec-9⁻ CD56^{dim} NK cells during HIV infection. Hinting at the potentially distinct role of Siglec-9, levels of Siglec-9⁺ CD56^{dim} NK cells, but not Siglec-7⁺ CD56^{dim} cells, inversely correlated with levels of CD4⁺ T cell-associated HIV DNA during suppressive ART. A functional analysis confirmed that sorted Siglec-9⁺ NK cells exhibit higher cytotoxicity towards HIV-infected cells compared to Siglec-9⁻ NK cells. These data are consistent with the highly cytotoxic nature of Siglec-9⁺ NK cells. However, blocking Siglec-9 enhanced the ability of NK cells to kill HIV-infected cells. This result is consistent with the known inhibitory function of the Siglec-9 molecule itself on these otherwise cytotoxic cells. These data support a model in which Siglec-9⁺ CD56^{dim} NK cells help in controlling HIV infection but are being restrained by the inhibitory nature of Siglec-9 receptor signaling.

The data highlight the Siglec-9/Sialic acid axis as a novel glyco-immune checkpoint mechanism that may be exploited by HIV-infected cells to evade immune surveillance by the cytotoxic Siglec-9⁺ NK cells.

The materials and methods used in the experiments are now described.

### Human Primary Peripheral Blood Mononuclear Cells (PBMCs).

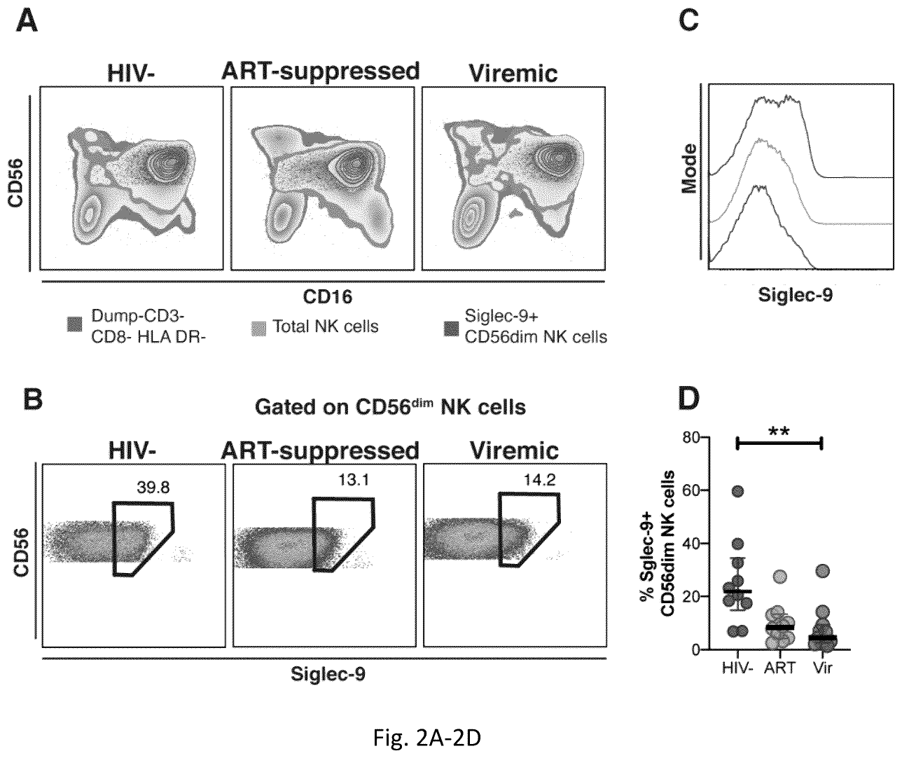

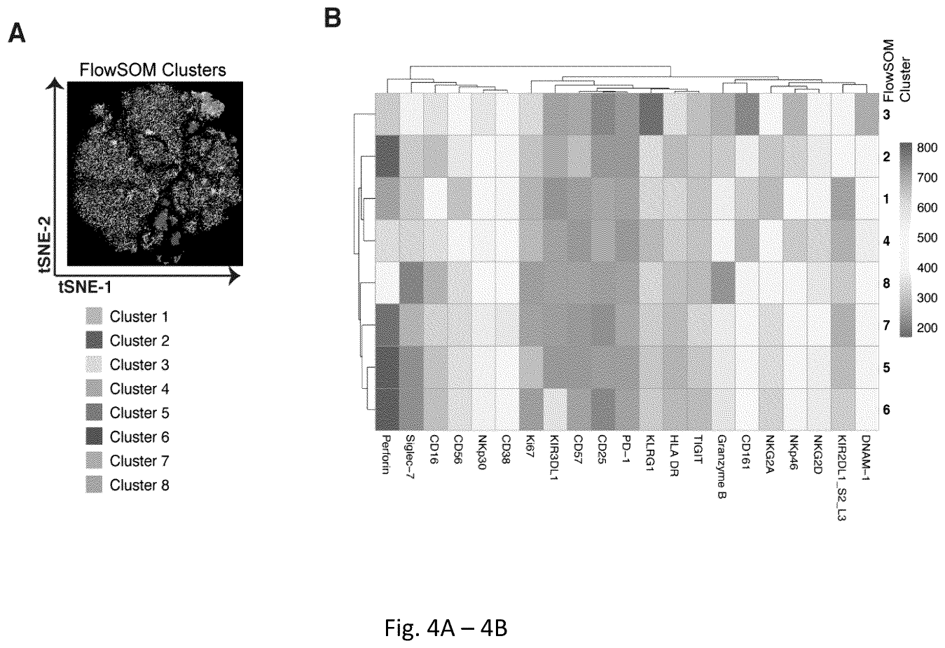

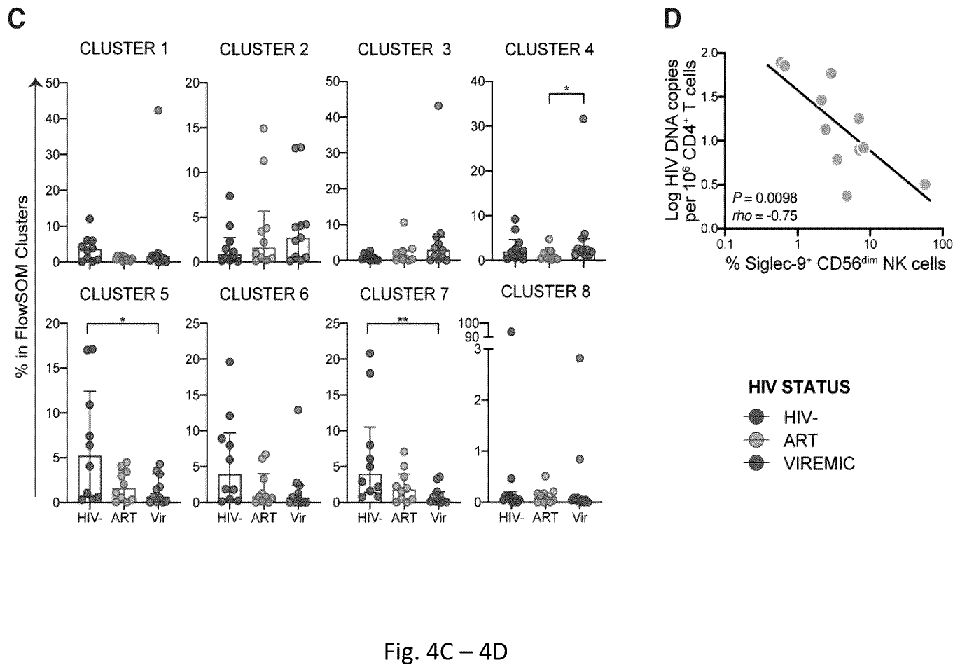

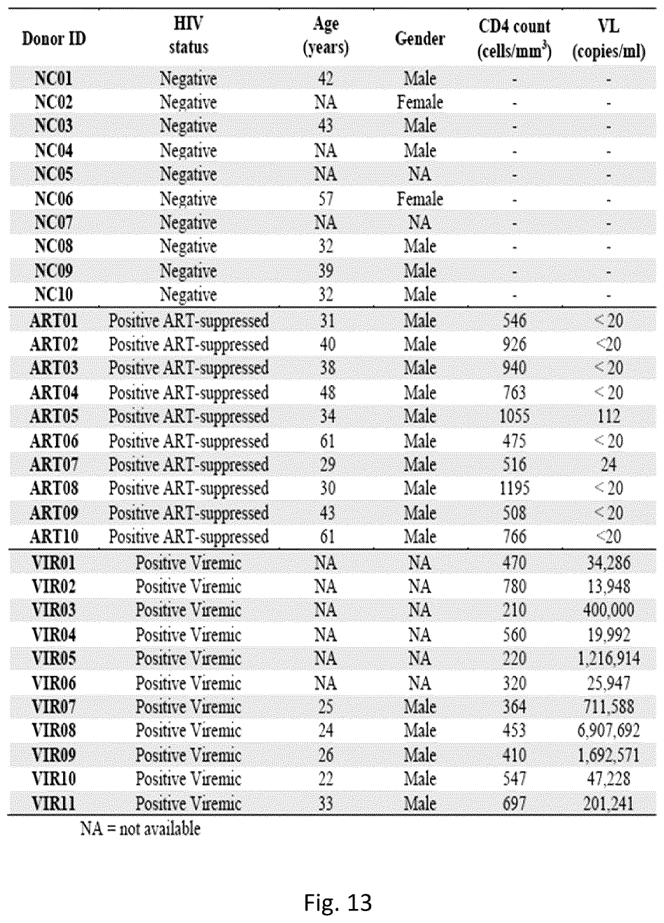

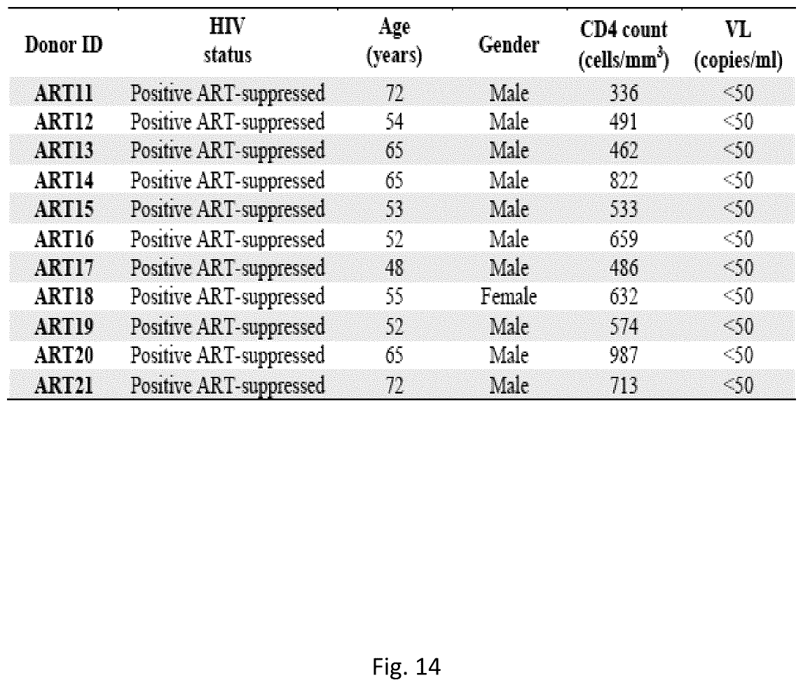

Siglec-9⁺ CD56^{dim} NK cells from 31 donors were phenotypically characterized (FIGS. 2, 3, and 4A-C): 10 HIV-negative controls; 11 HIV+ viremic; and 10 HIV+ on suppressive ART (clinical data of this cohort are in FIG. 13). Frozen PBMCs of the HIV-infected ART-suppressed individuals were obtained. Frozen PBMCs from HIV-negative and HIV-infected viremic individuals were obtained. The relationship between Siglec-9 expression on CD56^{dim} NK cells and levels of HIV DNA (FIG. 4C) using cells from 11 HIV-infected ART-suppressed individuals (clinical data of this cohort are in FIG. 14). Healthy HIV-negative PBMC samples were obtained.

Phenotypic Characterization of Siglec-9⁺ CD56^{dim} NK Cells in FIGS. 2, 3, 4A-C.

Phenotyping of NK cells expressing Siglec-9 was performed on cryopreserved PBMC from HIV−, HIV+ ART+ and HIV+ viremic individuals as previously published (Kuri-Cervantes et al., 2020, STAR Protoc 1, 100154). In brief, cryopreserved PBMC were thawed and rested at 2×10\hat{ }6 cells/ml for 3 hours in a complete R10 medium (RPMI 1640 supplemented with 10% FBS, 1% L-glutamine, and 1% penicillin/streptomycin) with 1 μl/ml of DNAse I (Roche, Branchburg, NJ) in the incubator at 37° C., 5% CO₂. After resting, 2×10\hat{ }6 PBMC/well were then plated into a 96 V-bottom well plate for staining. All staining steps were performed at room temperature in the dark. Cells were washed with PBS and resuspended in 45 μl/well of PBS. For viability exclusion, 5 μl/well of a 1:60 dilution of Live/Dead Fixable Aqua Dead Cell Stain Kit (Invitrogen) was added ad incubated for 10 minutes. The extracellular antibody cocktail was then added in a volume of 50 μl/well prepared in 1:1 solution of FACS buffer (0.1% sodium azide and 1% bovine serum albumin in 1×PBS) and BD Brilliant Stain buffer (BD Biosciences), reaching a final staining volume of 100 μl/well. The extracellular stain was incubated for 20 minutes. Cells were washed with FACS buffer and then fixed and permeabilized using the Foxp3/Transcription Factor Staining Buffer Set (eBioscience) following the manufacturer's instructions. The intracellular antibody cocktail was prepared in a final volume of 100 μl/well diluted 1× permwash buffer, added, and incubated for one hour. Finally, cells were washed with 1× permwash buffer and fixed with 1% paraformaldehyde. Cells were stored at 4° C. in the dark until acquisition on a BD FACS Symphony A5 flow cytometer (BD Biosciences) within 24 hours. All flow cytometry data were analyzed using FlowJo V10.7.1 software (Tree Star, Ashland, OR).

The following antibodies were used for staining, from BD Biosciences: TIGIT Brilliant Blue 700 (clone 741182), Ki-67 Alexa Fluor 700 (clone B56), HLA-DR Brilliant Ultra Violet 395 (clone G46-6), CD8 Brilliant Ultra Violet 496 (clone RPA-T8), CD16 Brilliant Ultra Violet 615 (clone 3G8), CD38 Brilliant Ultra Violet 661 (clone HIT2), CD25 Brilliant Ultra Violet 737 (clone DX12), CD3 Brilliant Ultra Violet 805 (clone UCHT1), NKP30 Brilliant Violet 480 (clone P30-15), KIR3DL1 Brilliant Violet 711 (clone DX9), Granzyme B PE-CF594 (clone GB11) and CD161 PE-Cy5 (clone DX12). From Biolegend: Siglec-9 APC (clone 351506), NKG2D APC-Cy7 (clone 1D11), Perform Brilliant Violet 421 (clone B-D48), CD14 Brilliant Violet 510 (clone M5E2), CD19 Brilliant Violet 510 (clone HIB19), CD56 Brilliant Violet 570 (clone HCD56), KLRG1 Brilliant Violet 605 (clone 2F1/KLRG1), NKP46 Brilliant Violet 650 (clone 9E2), PD-1 Brilliant Violet 750 (clone EH12.2H7), DNAM-1 Brilliant Violet 785 (clone 11A8), Siglec-7 PE (clone 339204), CD57 PE Cy7 (clone HNK-1). KIR2DL2/S2/L3 B PE Cy5.5 (clone GL183) was obtained from Beckman Coulter and NKG2A Alexa Fluor 488 (clone 131411) from R&D Systems. For viability exclusion, a Live/Dead Fixable Aqua Dead Cell Stain Kit (Invitrogen) was used for viability exclusion and was used following the manufacturer's instructions.

Phenotypic Characterization of Siglec-9⁺ CD56^{dim} NK Cells in FIG. 4D.

Cryopreserved PBMC were thawed in warm 10% cRPMI (RPMI 1640 medium; (Hyclone, Logan, Utah) supplemented with 10% fetal bovine serum (FBS) (Hyclone), 1% penicillin-streptomycin (Hyclone), 10 mM HEPES (Hyclone), 2 mM L-glutamine (Hyclone), and 10 μg/ml DNase I (Sigma-Aldrich, Dorset, United Kingdom), washed with PBS+2% FBS (Hyclone) (complete RPMI). Cells were stained for viability with an aqua amine reactive dye (AARD; Invitrogen), then incubated with panels of conjugated anti-human monoclonal antibodies (mAbs): CD3-ECD (clone UCHT1, Beckman Coulter), CD4-AF700 (clone RPA-T4, BD biosciences), CD8-Qdot 605 (clone 3B5, Invitrogen), CD14− APC-Cy7 (clone MφP9, BD biosciences), CD16-BV421 (clone 3G8, Biolegend), CD56-PE-Cy7 (clone B159, BD biosciences), CD161− FITC (clone HP-3G10, Biolegend), PD1− PerCP-Cy 5.5 (clone NAT105, Biolegend), Siglec-7-PE (clone 6-434, Biolegend), and Siglec-9-APC (clone 351506; Biolegend). Cells were then washed with PBS+2% FBS and then fixed in 1% paraformaldehyde (PFA, Electron Microscopy Sciences) before acquiring on a custom four laser LSRFortessa flow cytometer (BD Biosciences). Data were analyzed using Flowjo Software version 9.5 (Treestar).

qPCR Quantification of HIV DNA in Isolated CD4⁺ T Cells.

CD4⁺ T cells were isolated from the PBMCs of HIV-infected ART-suppressed individuals using the Human EasySep™ Human CD4⁺ T Cell Isolation Kit (StemCell Technologies). Isolated cells were lysed in RLT Plus Buffer (Allprep isolation kit, Qiagen). Total DNA was extracted from the lysates using the Allprep DNA/RNA/miRNA Universal Kit (Qiagen). Total HIV DNA was quantified with a qPCR TaqMan assay using LTR-specific primers F522-43 (5′-GCCTCAATAAAGCTTGCCTTGA-3′, SEQ ID NO:1; HXB2522-543) and R626-43 (5′-GGGCGCCACTGCTAGAGA-3′, SEQ ID NO:2; 626-643) coupled with a FAM-BQ probe (5′-CCAGAGTCACACAACAGACGGGCACA-3′, SEQ ID NO:3) (Kumar et al., 2007, J Neurovirol. 13, 210-224) using the StepOne Plus Real-Time PCR System (Applied Biosystems). Cell-associated HIV DNA copy number was determined using a reaction volume of 20 μl with 10 μl of 2× TaqMan Universal Master Mix II, including UNG (Applied Biosystems), 4 pmol of each primer, 4 pmol of the probe, and 5 μl of DNA. Cycling conditions were 50° C. for 2 min, 95° C. for 10 min, followed by 60 cycles of 95° C. for 15s and 59° C. for 1 min. External quantitation standards were prepared from DNA isolated from ACH-2 cells in a background of HIV-1 negative human cellular DNA, calibrated to the Virology Quality Assurance (VQA, NIH Division of AIDS) cellular DNA quantitation standards. Cell counts were determined by qPCR using human genomic TERT (Applied Biosystems). Copy number was determined by extrapolation against a 7-point standard curve (1-10,000 copies) performed in triplicate.

### Cell Culture.

HUT78, HUT78/SF2, CEM×174, CEM.NKR and CEM.NKR CCR5+ Luc+ cells were obtained through the NIH HIV at 37° C. with Reagent Program, Division of AIDS, NIAID, NIH and cultured in RPMI 1640 supplemented with heat-inactivated 10% fetal bovine serum (FBS), L-glutamine (2 mM), penicillin (50 U/ml), and streptomycin (50 mg/ml). Cultures were grown in T75 flasks (Thermo Fisher) and maintained 5% CO2. Expi293F cells (Thermo Fisher) were maintained in Expi293 expression medium at 37° C. with 8% CO2.

### HIV Infection of CEM.NKR and CEM.NKR CCR5⁺ Luc+ Cells.

2 ml solution of HIV-1 HXB2 (873,464 TCID50/ml) or DH12 (667,959 TCID50/ml) grown in CEM×174 cells were added to RetroNectin precoated dish (Takara Bio) and incubated at 37° C. for 6 hours. Following incubation, virus solution was removed from the dish and 5×105 cells CEM.NKR or CEM.NKR CCR5+ Luc+ cells were added. After a 72 h incubation at 37° C., cells were washed and used for downstream assays.

### Cell Surface Gp120 Stain.

Anti-gp120 antibody binding to HIV-infected cells was evaluated by flow cytometry. Indicated antibodies at 20 μg/mL were incubated with 2×105 HUT78/SF2 cells in 100 μL PBS supplemented with 0.5% bovine serum albumin 0.1% sodium azide and incubated for 30 minutes at room temperature. After washing twice, cells were resuspended in 100 μL PE Fc-specific goat anti-human IgG (eBiosceince); 1:250 dilution, for 20 min at room temperature. Cells were again washed, fixed, and analyzed by flow cytometry.

### p24 Intracellular Stain.

To evaluate HIV infection, 2×105 cells were pelleted and resuspended in fixation buffer (BD Cytofix/Cytoperm) for 20 min at 4° C. After fixation, cells were permeabilized by washing twice with permeabilization buffer (BD Perm/Wash Buffer). Cells were resuspended in 100 μl permeabilization buffer, and 2.5 μl of PE-conjugated anti-p24 Ab KC57 (Beckman Coulter) was added and incubated for 30 min at room temperature. After incubation, cells were washed once with permeabilization buffer and with PBS supplemented with 0.5% bovine serum albumin 0.1% sodium azide a second time and analyzed by flow cytometry.

### Primary Human NK Cell Isolation.

NK cells were isolated from peripheral blood mononuclear cells (PBMC) obtained from healthy donors by immunomagnetic negative selection using the EasySep Human NK Cell Isolation Kit (STEMCELL Technologies) following the manufacturer's protocol. Isolated cells were used immediately for cytotoxicity assays.

### Depletion of Siglec-9+NK Cells.

2.5 ug human Siglec-9 biotinylated antibody (R&D Systems) was combined with 1×106 purified NK cells and incubated for 10 min at 4° C. Excess antibodies were removed by washing twice with PBS supplemented with 0.5% BSA. Cells were further incubated with 25 μl streptavidin MicroBeads (Miltenyi Biotec) for 15 min at 4° C. Cells were washed once and loaded onto pre-equilibrated LS columns (Miltenyi Biotec) according to the manufacturer's instructions. After washing extensively, cells in the eluate fraction were pelleted and resuspended in a suitable medium for downstream assay.

### Sorting of Siglec-9+ and Siglec-9− NK Cells.

Isolated human NK cells were resuspended PBS buffer supplemented with 0.5% bovine serum albumin and stained with anti-CD3 BV510 (BD), anti-CD56 PerCP Cy5.5 (BD), and anti-Siglec-9 PE (BioLegend) for 15 min at 4° C. Cells were washed twice and sorted using the MoFlo Astrios EQ, cell sorter (Beckman Coulter). Sorted cells were gated on the CD3− CD56dim population.

### NK Cytotoxicity Assay by Lactate Dehydrogenase (LDH) Release.

This is a measure of endogenous lactate dehydrogenase (LDH) released from target cells in the presence of effector NK cells. Indicated target cells were resuspended in serum-free RPMI medium at a concentration of 2×105 cells/ml. 2×104 target cells were plated in a 96-well V-bottom plate (Corning) on 100 μl RPMI. Next, 2×105 isolated effector NK cells resuspended in RPMI were added to the targets at an effector-to-target ratio of 10:1. Control wells were adjusted to equal volumes. Cells were mixed, pelleted at 200 g for 2 min, and incubated at 37° C. for 16 h. Following incubation, 10 μl of supernatant was diluted 1:10 in LDH storage buffer in a 96-well round-bottom microplate following the LDH-Glo Cytotoxicity Assay protocol (Promega). Samples were further diluted two-fold in the LDH detection reagent. Luminescence was recorded with a plate reader after a 60-minute incubation at room temperature. Percent cytotoxicity was calculated as: ((experimental RLU−effector spontaneous control RLU−target cell spontaneous control RLU)/(Target cell maximum control RLU−target cell spontaneous control RLU))×100. In experiments that involved siglec-9 antibodies, purified human NK cells were pretreated with anti-Siglec-9 antibody or isotype-matched control antibody for 1 h at 37° C. in 96-well microplates before the addition of indicated target cells. Volumes of test and control wells were adjusted to be equal.

### CFSE/SYTOX Red NK Direct Cytotoxicity Assay.

In this method, target cells are tracked with fluorescent dyes to distinguish them from unlabeled effectors. 5×105 target cells in 500 μl RPMI were labeled with 2 μM of the green fluorescent dye carboxyfluorescein diacetate succinimidyl ester (CFSE) (Life Technologies) for 1 h at 37° C. Following incubation, labeling reaction was quenched with 10 ml complete growth medium—RPMI 1640 supplemented with heat-inactivated 10% fetal bovine serum (FBS), L-glutamine (2 mM), penicillin (50 U/ml), and streptomycin (50 mg/ml). Labeled target cells were resuspended in a complete growth medium at a concentration of 2×105 cells/ml. 2×104 target cells were plated in a 96-well V-bottom plate (Corning) on 100 μl complete growth medium. Next, 2×105 isolated effector NK cells resuspended in complete growth medium were added to the targets at an effector-to-target ratio of 10:1. Cells were mixed, pelleted at 200 g for 2 min, and incubated at 37° C. for 16 h. Control wells were adjusted to equal volumes with a complete growth medium. Following incubation, 50 μl SYTOX Red is added to wells for a final concentration of 5 nM. Cells are analyzed by flow cytometry. The FITC channel was used to capture CFSE+ events and APC channel, SYTOX Red+ events. Percentage target cell death was calculated as the ((FITC+ APC+ events)/(FITC+ APC− events))×100.

### NK Degranulation and Cytokine Production Assay.

Indicated target cells were combined with purified human NK cells at an effector-to-target ratio of 4:1 or 2.5:1 (as indicated) in a complete growth medium in the presence of GolgiStop (BD) and anti-CD107a PE antibody (BD). The cell mixture was pelleted at 200 g for 2 min and incubated at 37° C. for 16 h. Post incubation, cells were stained for surface markers with anti-CD56 PerCP Cy5.5 (BD) and anti-CD3 BV510 (BD). Cells were washed twice, fixed (BD Cytofix/Cytoperm), and permeabilized (BD Perm/Wash buffer). Following permeabilization, anti-IFN-gamma BV421 (BD) antibody was used for intracellular staining. All assays were acquired via flow cytometry with an LSR II. NK cells were defined as CD3− and CD56⁺. Data were reported as the percentage of cells positive for CD107a and/or IFN-gamma. In experiments that involved siglec-9 antibodies, purified human NK cells were pretreated with anti-Siglec-9 antibody or isotype-matched control antibody for 1 h at 37° C. in 96-well microplates before the addition of indicated target cells.

### Detection of Cell Surface Siglec-9 Ligands.

1×10⁵ cells were resuspended in 100 μl PBS supplemented with 0.5% bovine serum albumin 0.1% sodium azide. 1 μg recombinant human Siglec-9-Fc (R&D Systems) was added and incubated for 1 h at room temperature. Following incubation, cells were washed twice with PBS supplemented with 0.5% bovine serum albumin 0.1% sodium azide and incubated with PE Fc-specific goat anti-human IgG (eBiosceince); 1:250 dilution, for 20 min at room temperature. Cells were further washed twice, fixed (BD Cytofix/Cytoperm), and acquired by flow cytometry (LSR II, BD). Flow cytometry data were analyzed using FlowJo software.

Siglec-9 Ligand Detection after Desialylating Cells.

1×10⁵ cells were resuspended in 100 μl PBS supplemented with 0.5% bovine serum albumin 0.1% sodium azide. 100 nM ST sialidase was added and incubated for 1 h at 37° C. To remove residual ST sialidase, cells were washed twice by centrifugation 400 g for 5 min. Next, cells were evaluated for cell surface Siglec-9 ligand content following protocol to detect cell surface siglec-9 ligands as described.

HIV bNAbs Expression and Purification.

To produce recombinant anti-gp120 mAbs, Expi293F cells were transfected with plasmid DNA using Expifectamine following the manufacturer's protocol for (Thermo Scientific). 18 h after transfection, enhancers were added to the cultures. Antibody-containing supernatants were harvested four days post-transfection, clarified at 1,000 g for 10 min at 4° C. Supernatants were further then filtered through a 0.45-μm filter unit (Fisher Scientific). To purify IgG from the supernatants, protein G MagBeads (GenScript) was then used following the manufacturer's protocol. Bound antibodies were separated using magnets, and eluted with eluted antibodies were filtered through a 0.2-μm filter unit. The concentrations of purified recombinant antibodies were determined using a NanoDrop by measuring absorbance at A280.

Proximity-Induced Antibody Conjugation (pClick)—to Conjugate HIV bNAbs to Sialidase.

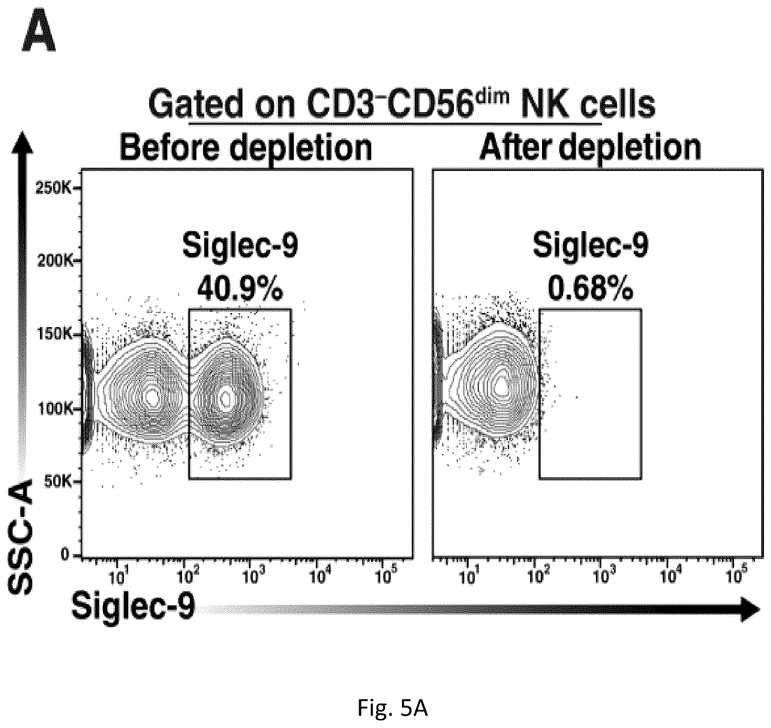

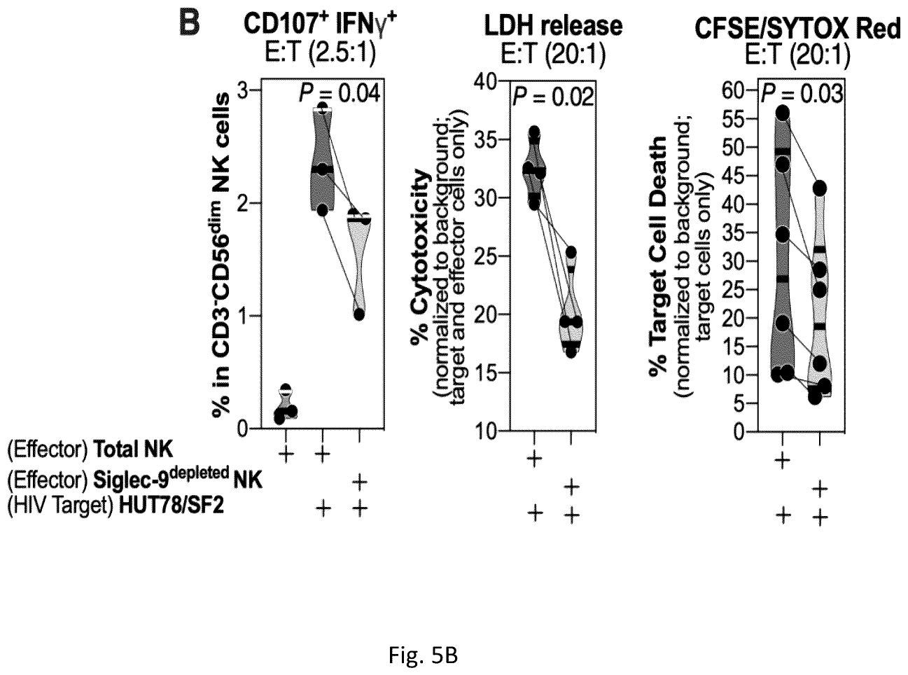

pClick enables site-specific covalent bond formation between functional moieties and native antibodies without antibody engineering, UV, or chemical treatment. In pClick, non-canonical amino acids (ncAAs), which allow crosslinking to a proximal lysine residue, are introduced into specific sites of affinity peptides (FB peptide) that have well-characterized binding sites within the fragment crystallizable or antigen-binding fragment of the antibody. Upon binding to the antibody, the ncAA-containing FB peptide enables proximity-induced covalent attachment to the nearby lysine residue of the antibody (FIG. 5A top). To site-specifically conjugate the HIV bNAbs with Sialidase (from Salmonella typhimurium) using pClick, 4-fluorophenyl carbamate lysine was genetically incorporated into the Glu25 position of a FB fused with Sialidase using the genetic code expansion technology. Next, bNAb-Sia conjugates were prepared by incubating bNAbs with 16 equivalents of FB-Sia for 48 h. The resulting bNAb-Sia conjugates were then purified by size-exclusion column. The conjugates were characterized by SDS-PAGE and determined an enzyme/antibody ratio=1.0.

### Antibody-ST-Sia Conjugate Selectivity.

An equal number of HUT78 and HUT78/SF2 cells were mixed and then treated with antibody-ST-Sia conjugates of unconjugated anti-gp120 antibodies only for 1 h at 37° C. Following incubation, cells were washed twice with PBS supplemented with 0.5% bovine serum albumin 0.1% sodium azide and costained with FITC-labeled-SNA and PE Fc-specific goat anti-human IgG (eBiosceince) for 30 min at room temperature. Cells were washed, fixed, and analyzed by flow cytometry. Cell surface sialylation levels were revealed with SNA-FITC, and gp120 levels were determined using anti-gp120-specific antibodies.

CEM.NKR CCR5+ Luc+ ADCC assay.

CEM.NKR CCR5+ Luc+ is a cell line derived from CEM.NKR CCR5+ that stably expresses the luciferase reporter gene under the transcriptional control of the HIV LTR. Upon HIV infection of these cells, Tat drives expression of luciferase, which can be quantified in the presence of a suitable substrate. HIV-infected and uninfected CEM.NKR CCR5+ Luc+ cells were washed with PBS and resuspended in RPMI. Cells were plated at 2×104 cells/well in a V-bottom microplate (Corning). HIV-infected CEM.NKR CCR5+ Luc+ were treated with indicated concentrations of anti-gp120 antibodies or Ab-ST-Sia conjugates for 2 h at 37° C. Control wells without antibodies were adjusted to volume with RPMI. Following incubation, purified human NK cells were added to wells at an effector-to-target ratio (E:T) of 10:1. Cells were mixed pelleted at 200 g for 2 min and incubated for 16 h at 37° C. Following incubation, 10 μl of supernatants were subject to LDH analysis. To evaluate luciferase activity, 100 μl supernatant was removed from all wells and replaced with 100 μl Bright-Glo luciferase substrate reagent (Promega). After 2 min, the well contents were mixed and transferred to a clear-bottom black 96-well microplate. Luminescence (RLU) measurements were integrated over 1 second per well. Raw RLU values are shown relative to the light output generated in RPMI medium only (background).

### Statistical Analysis.

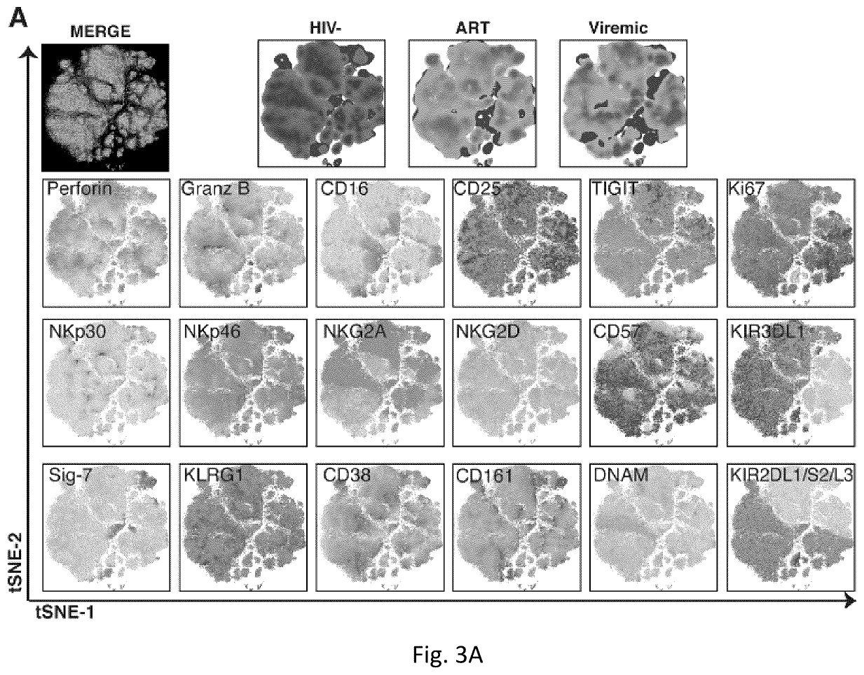

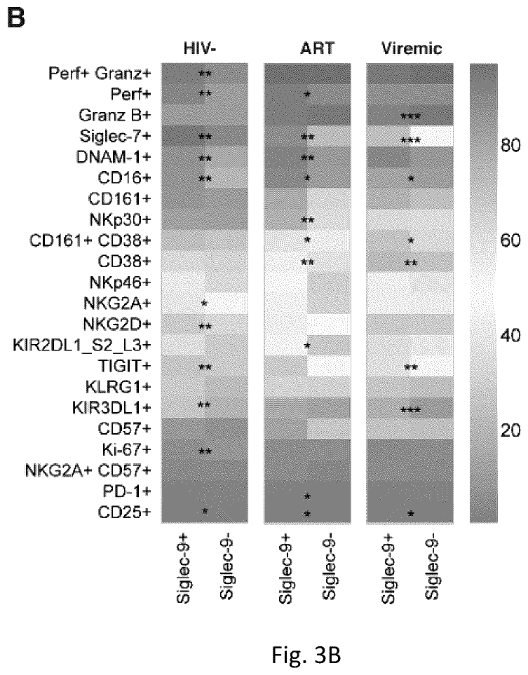

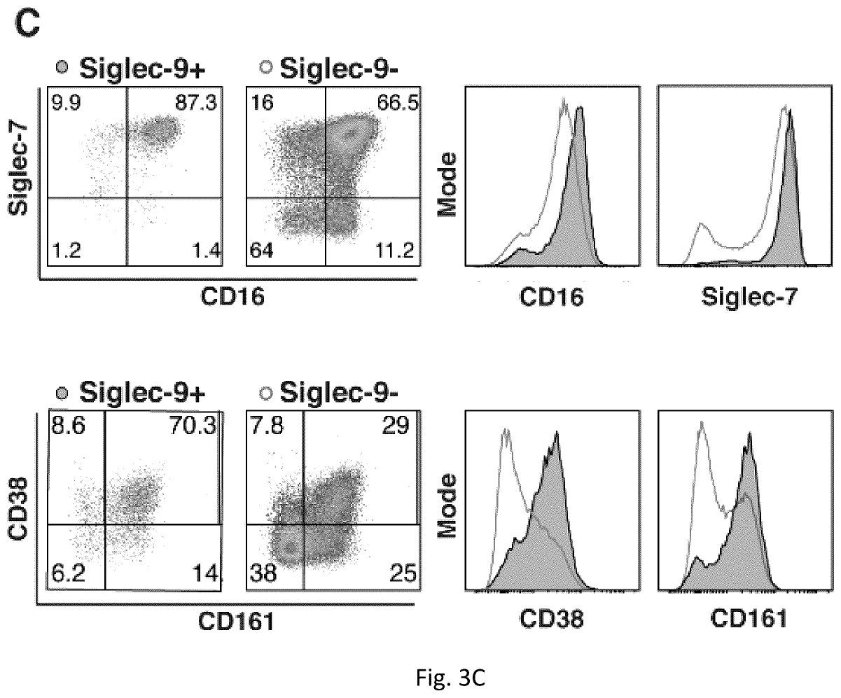

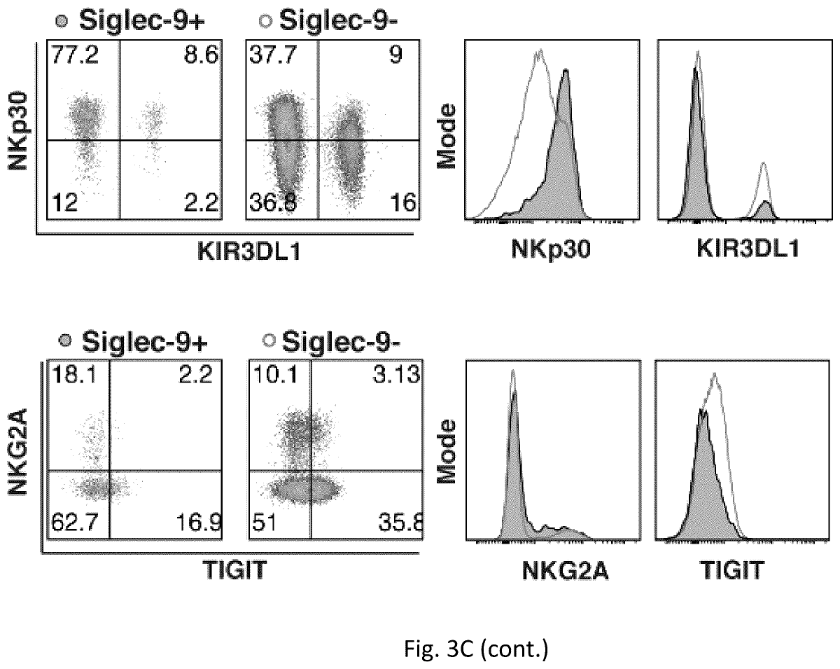

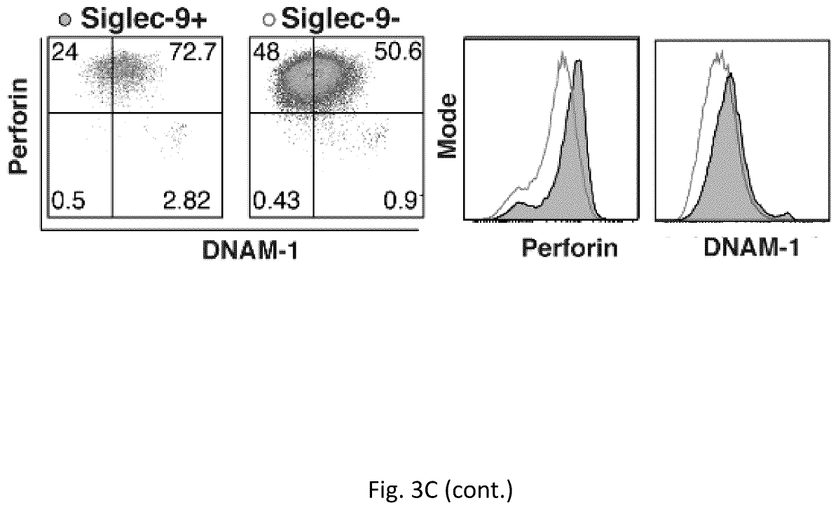

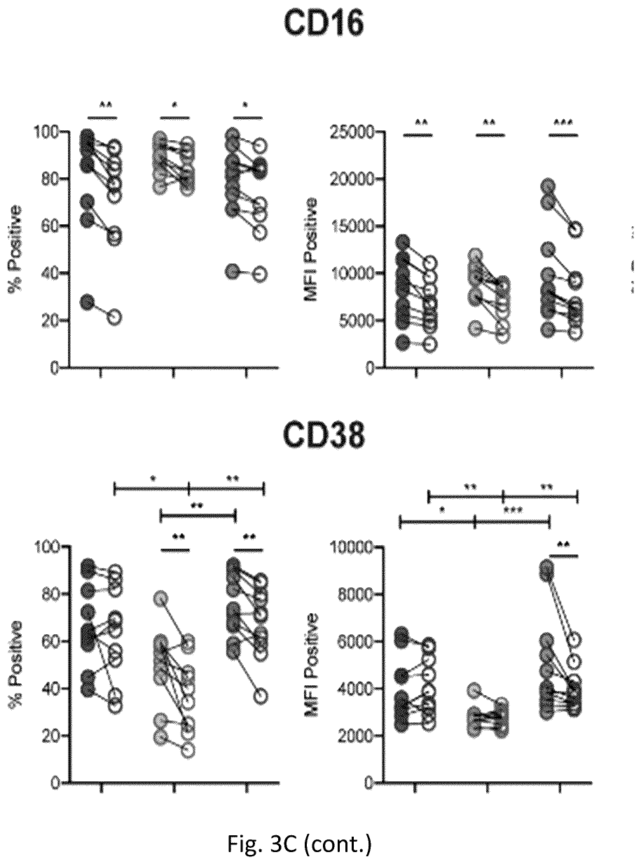

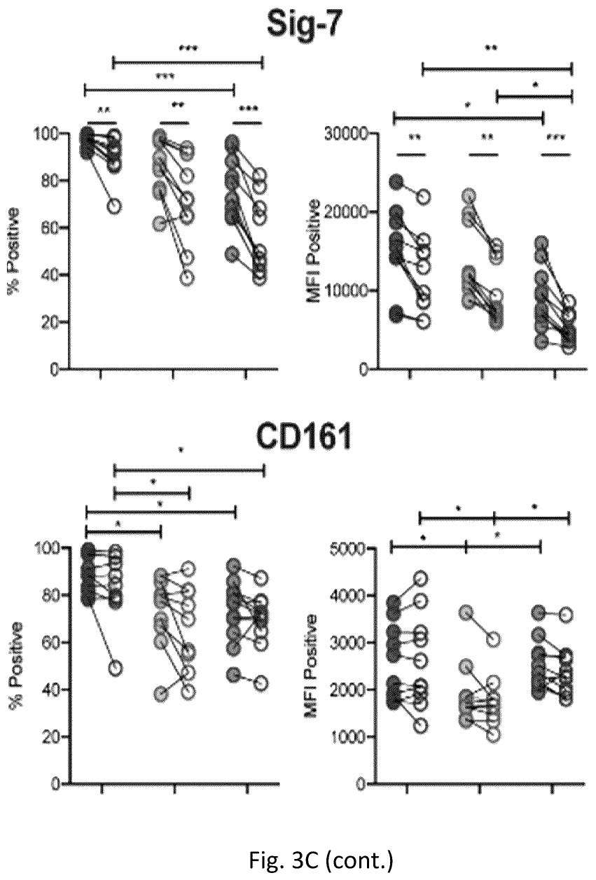

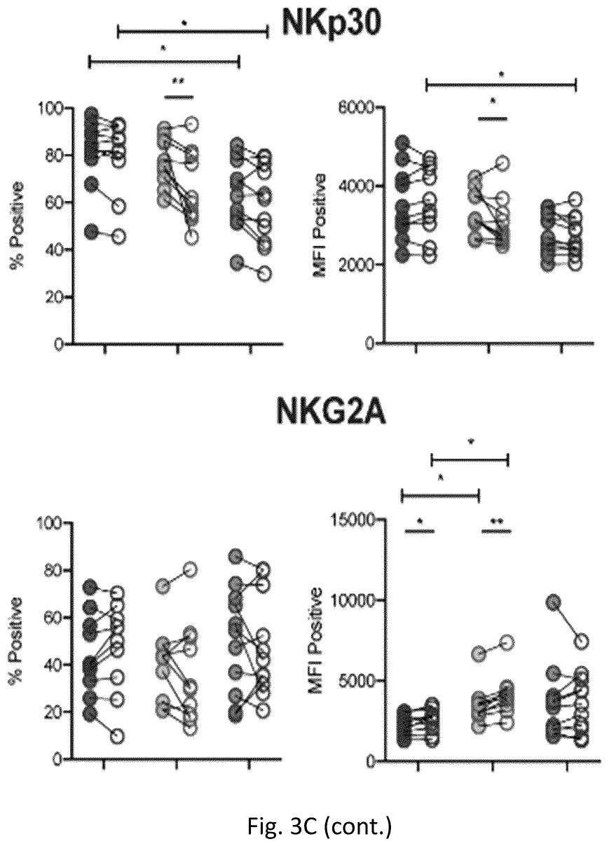

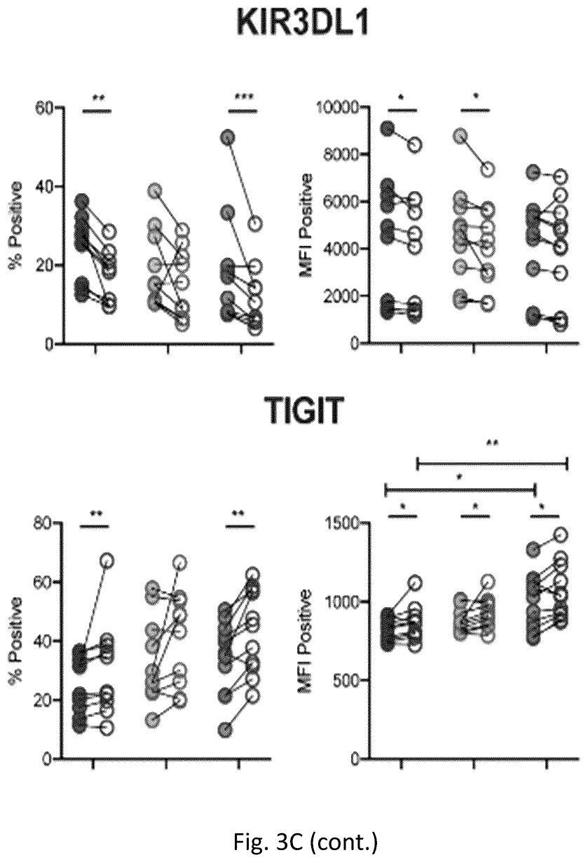

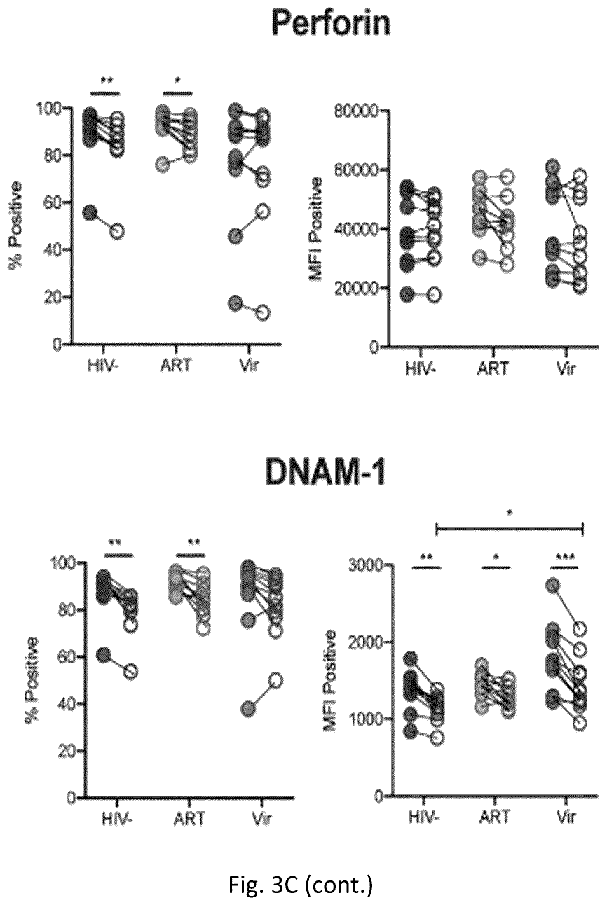

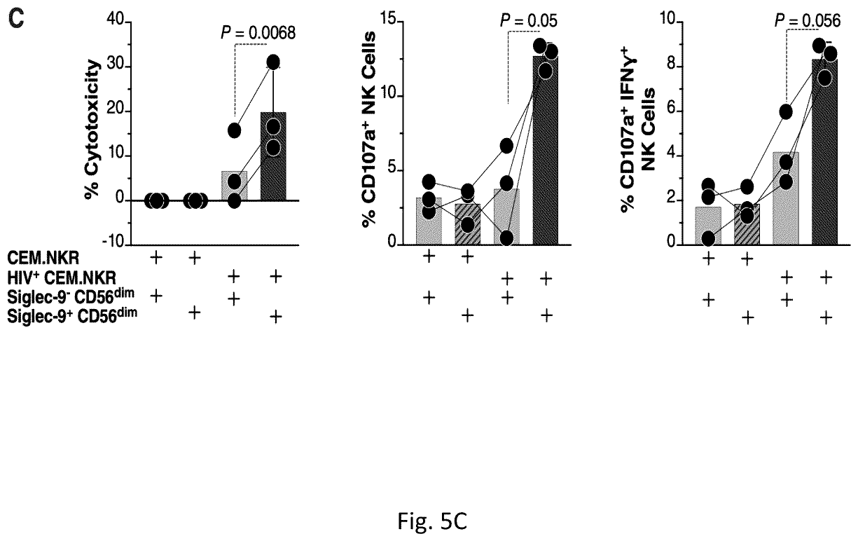

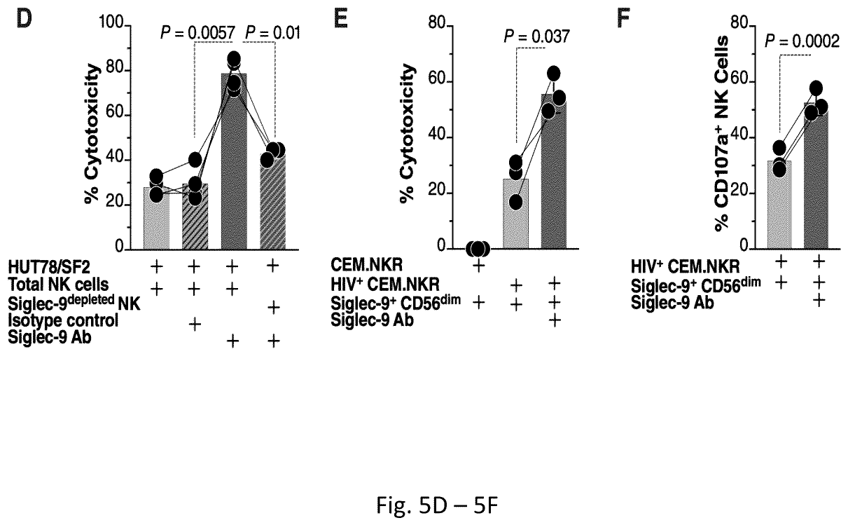

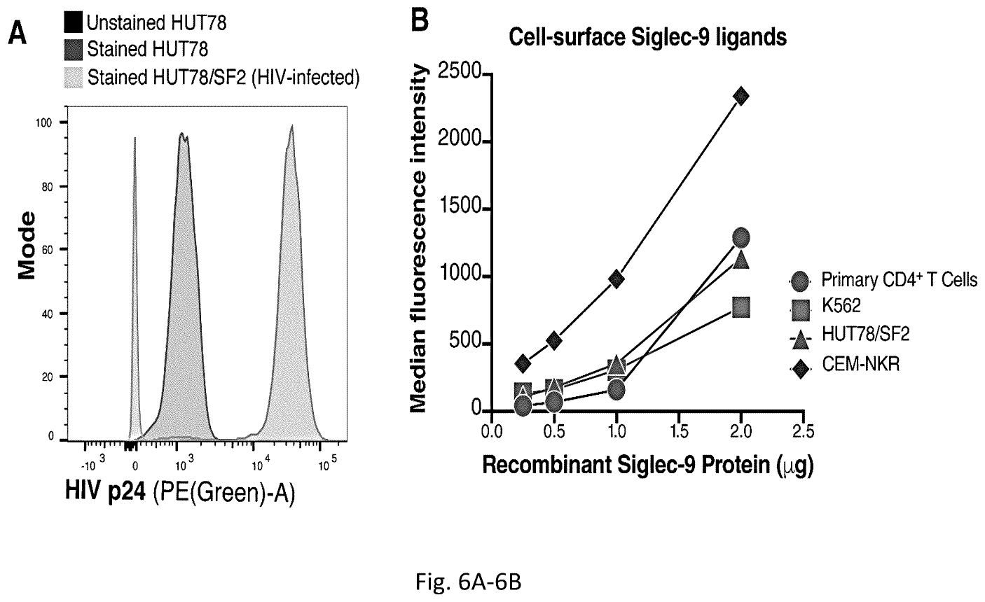

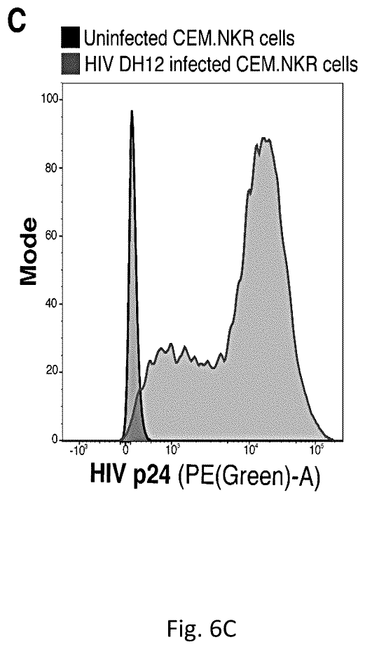

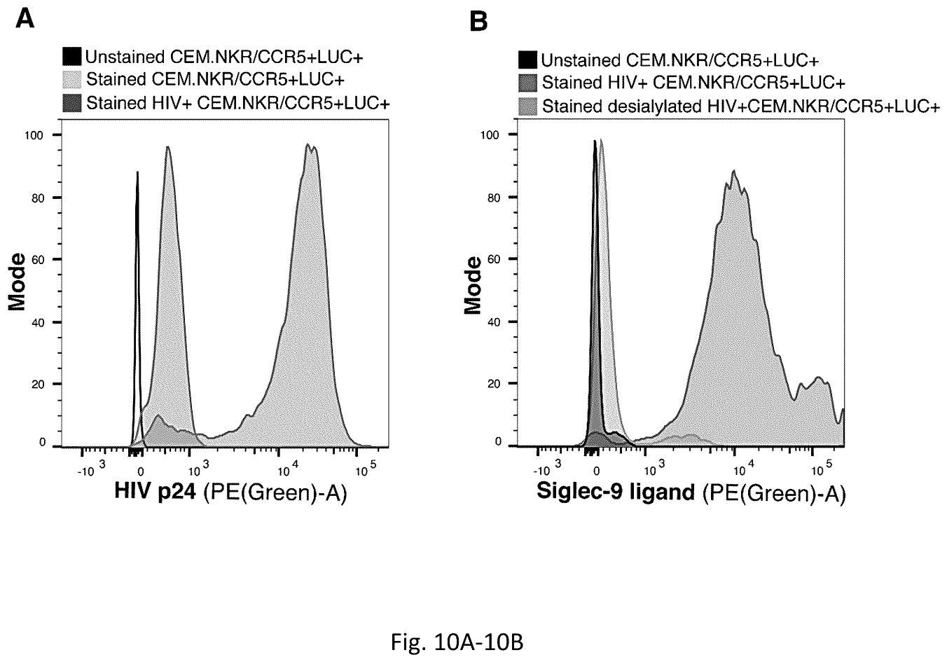

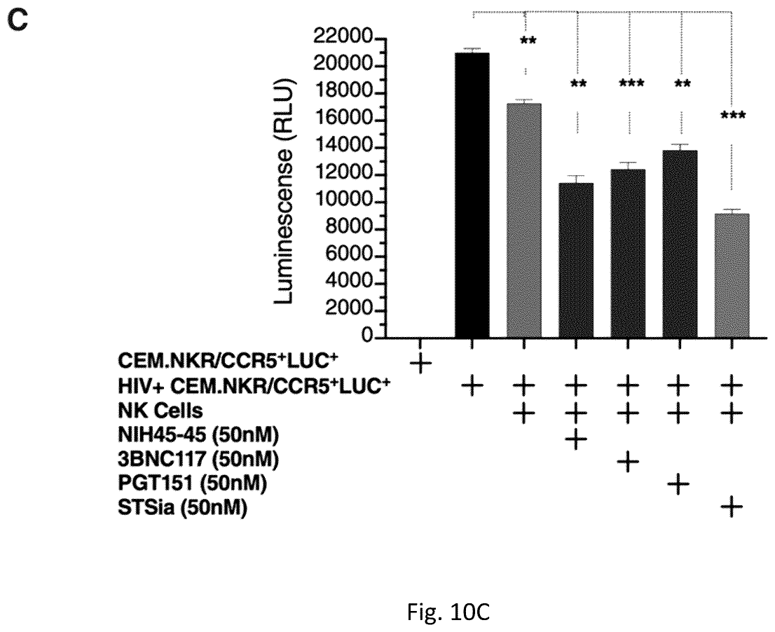

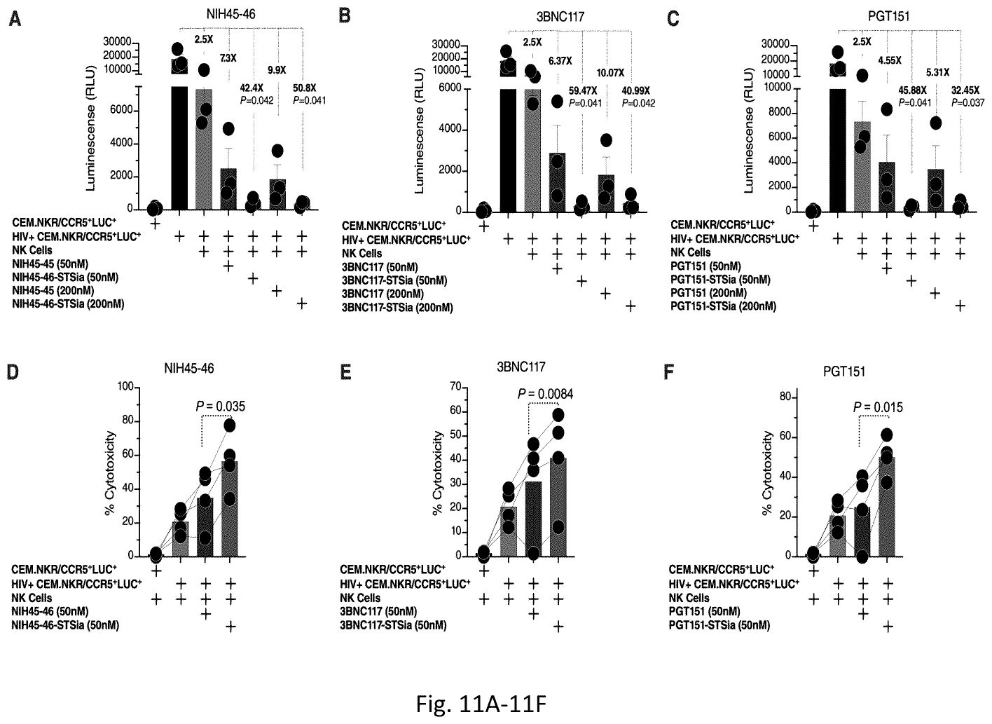

Data were analyzed using Prism 7.0 (GraphPad Software). Mann-Whitney rank tests were used for the analyses in FIG. 2D, FIG. 3C (between groups), and FIG. 4C used to compare between groups. Paired Wilcoxson tests were used to compare between Siglec-9+ and Siglec-9− within each group in FIG. 3B-C. Spearman's rank correlation was used in FIG. 4D. Paired t-tests were used for analyses in FIG. 5 and FIG. 6). Finally, a one-way ANOVA test was used in FIG. 11 and FIG. 10C.

The experimental results are now described.

### Siglec-9 is Expressed on a Subset of Activated CD56^{dim} NK Cells During HIV Infection.

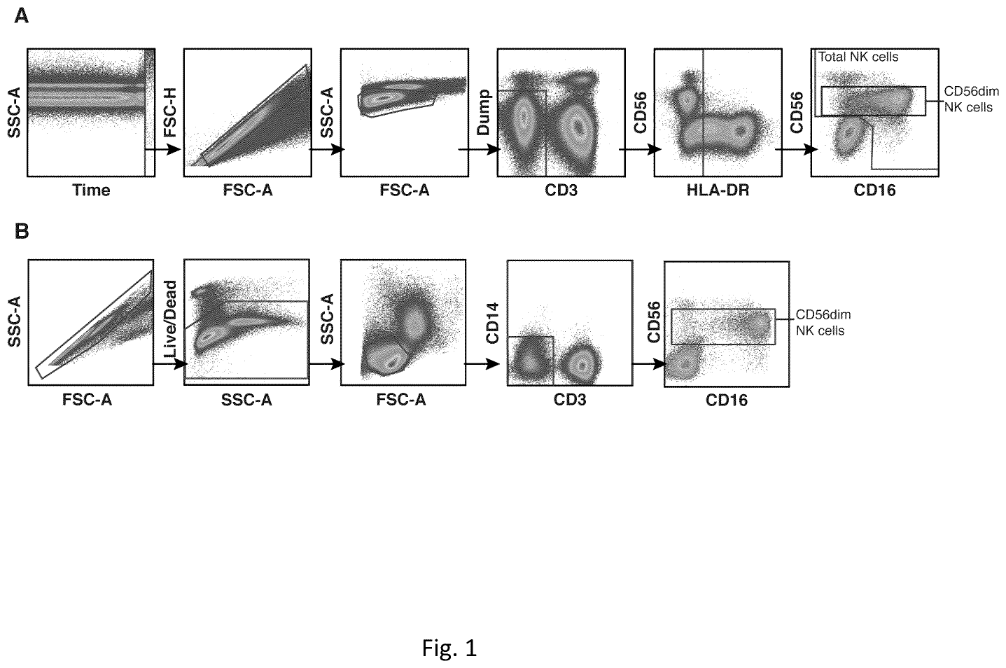

A decreased level of Siglec-7 has been described as a marker for a dysfunctional NK subset in HIV viremic individuals (Brunetta et al., 2009, Blood 114, 3822-3830; Varchetta et al., 2013, Retrovirology 10, 154; Zulu et al., 2017, AIDS Res Hum Retroviruses 33, 1205-1213). However, the role of Siglec-9 in HIV infection has not been elucidated. First, the cell-surface expression of Siglec-9 on NK cells was characterized to determine whether Siglec-9 expression levels differed between HIV+(ART-suppressed or viremic) individuals and HIV-negative controls (clinical data of this cohort are in Table 1). A comprehensive 27-color phenotypic analysis was performed (FIG. 1) of Siglec-9⁺ CD56^{dim} NK cells. It was found that Siglec-9 is expressed on a subset of CD56^{dim} NK cells regardless of HIV status (FIG. 2A). Next, it was found that the levels of Siglec-9⁺ CD56^{dim} NK cells are significantly lower in HIV+ individuals (viremic or ART-suppressed) compared to HIV-negative controls (FIG. 2B-D). These data suggest that HIV infection, regardless of the disease status. is associated with a depletion of the Siglec-9⁺ CD56^{dim} NK cells.

The cytotoxic potential of NK cells is regulated by a collection of activating and inhibitory signals delivered by cell surface receptors (Vivier et al., 2011, Science 331, 44-49). Therefore, Siglec-9⁺ CD56^{dim} NK cells were evaluated for their expression of activation and inhibitory receptors/markers during HIV infection. The expression of 18 markers was evaluated on Siglec-9⁺ CD56^{dim} NK cells (FIG. 3A). The expression of these markers on Siglec-9⁺ versus Siglec-9⁻ CD56^{dim} NK cells was compared (FIG. 3B-C). Siglec-9⁺ CD56^{dim} NK cells, during HIV infection, exhibit higher expression of several NK activating/cytotoxic receptors and markers including CD16 (% and mean fluorescence intensity (MFI)), CD38 (% and MFI), NKp30 (% and MFI), DNAM-1 (% and MFI), and perforin (%); and lower expression of the inhibitory receptor NKG2A (MFI) and TIGIT (% and MFI), compared to Siglec-9⁻ CD56^{dim} NK cells (FIG. 3B-C). However, Siglec-9⁺ CD56^{dim} NK cells also express higher levels of the inhibitory markers Siglec-7 (% and MFI) and KIR3DL1 (% and MFI). These data suggest that Siglec-9 marks a distinct subpopulation of NK cells during HIV infection. This subpopulation is characterized by a high expression of several NK activating receptors and markers and a differential expression of several inhibitory receptors and markers. These data are in agreement with existing literature from cancer and HBV fields that the Siglec-9⁺ CD56^{dim} NK subpopulation harbors a mature and activated phenotype (Jandus et al., 2014, The Journal of clinical investigation 124, 1810-1820; Zhao et al., 2018, Frontiers in immunology 9, 1124).

Siglec-9⁺ CD56^{dim} subpopulation is composed of different cell clusters.

Next, whether the Siglec-9⁺ CD56^{dim} subpopulation is composed of different cell clusters was examined. Using FlowSOM clustering (Van Gassen et al., 2015, Cytometry A 87, 636-645), eight different clusters of Siglec-9⁺ CD56^{dim} NK cells were identified (FIG. 4A), based on differential expression of NK activating and inhibitory receptors and markers (FIG. 4B). Of these different clusters, clusters 5 and 7 are particularly decreased during HIV infection (FIG. 4C). These two clusters are characterized by higher expression of the activating markers perform, NKp30, CD16, and Ki67 and lower expression of the inhibitory receptor NKG2A compared with other clusters (FIG. 4B). These data suggest that HIV infection (regardless of treatment status) specifically depletes several clusters of Siglec-9⁺ CD56^{dim} NK cells that express high levels of NK activating receptors and markers.

Siglec-9⁺ CD56^{dim} Cell Levels Correlate with Lower HIV DNA During ART.

Given the potentially activated phenotype of Siglec-9⁺ CD56^{dim} NK cells during ART-suppressed HIV infection, the relationship between Siglec-9 expression on NK cells and total HIV DNA measured in CD4⁺ T cells from 11 HIV+ ART+ individuals was examined (clinical data of this cohort are in Table 2). It was found that the percentage of Siglec-9⁺ CD56^{dim} NK cells correlated with lower levels of CD4⁺ T cell-associated total HIV DNA (FIG. 4D). In contrast, significant correlations were not observed between the percentage of Siglec-7 on CD56^{dim} or CD56^{bright} NK cells and total HIV DNA. These data, together with data in FIGS. 2, 3, are consistent with the notion that Siglec-9⁺ CD56^{dim} NK cells are cytotoxic and likely play a role in controlling viral infections (Zhao et al., 2018, Frontiers in immunology 9, 1124), including HIV infection.

Siglec-9^{depleted} NK Cells Exhibited Lower Cytotoxicity Towards HIV⁺ Cells Compared to Total NK Cells.

The phenotypic data in FIGS. 2-4 suggest that Siglec-9⁺ NK cells may be highly cytotoxic. To test this, the cytotoxicity of total NK cells depleted of Siglec-9⁺ CD56^{dim} NK cells (Siglec-9^{depleted} NK cells) was compared to the cytotoxicity of total NK against HIV+ cell lines. NK cells were isolated from 3-6 HIV-uninfected donors and the Siglec-9⁺ NK cells were depleted (FIG. 5A). The cytotoxicity of total NK cells and Siglec-9^{depleted} NK cells was then compared against a T cell line, HUT78 infected with SF2 HIV (dual tropic virus) (HUT78/SF2; FIG. 6A). These T cells have levels of Siglec-9 ligands (α2-3 Sialic acid) comparable to primary human CD4⁺ T cells (FIG. 6B). Assays were performed in triplicate wells for each donor, and an average was used for statistical analysis. Cytotoxicity was assessed using three different measures: (1) NK degranulation [frequency of CD56^{dim} NK cells expressing CD107a and IFN-γ; by flow cytometry (Tomescu et al., 2017, Aids 31, 613-622; Papasavvas et al., 2017, J Viral Hepat 24, 865-876; Tomescu et al., 2007, Journal of immunology 179, 2097-2104)]; (2) Levels of lactate dehydrogenase (LDH) released into the supernatant from damaged cells [normalized to background using target cells and effector cells alone; by luminescence assay]; and (3) the proportion of lysed target cells using the CFSE/SYTOX method (Kandarian et al., 2017, J Vis Exp (126):56191). In the CFSE/SYTOX method, target cells were pre-labeled with CFSE dye. After co-culturing effector and target cells, killed target cells were identified by SYTOX Red stain, which selectively permeates dead cells. Cytotoxicity was measured as the proportion of dead target cells (SYTOX Red⁺ CFSE⁺) to the total number of targets (CFSE⁺); by flow; normalized to target cells only (Gomez-Roman et al., 2006, J Immunol Methods 308, 53-67). Results from each measure show that total NK cells exhibit higher cytotoxicity than Siglec-9^{depleted} a NK cells (FIG. 5D). These data suggest that the Siglec-9⁺ CD56^{dim} subpopulation of NK is an important contributor to NK cytotoxicity against HIV-infected cells.

Siglec-9⁺ NK Cells Exhibited Higher Cytotoxicity Towards HIV+ Cells Compared to Siglec-9⁻ NK Cells.

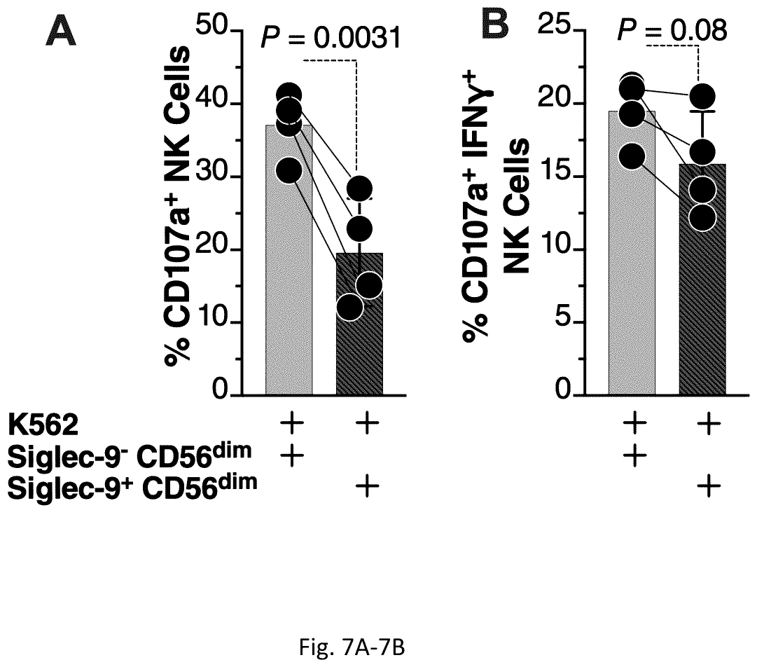

Next Siglec-9⁺ and Siglec-9⁻ CD56^{dim} NK were sorted (using fluorescence-activated cell sorting (FACS)) from three healthy donors (assays were done in triplicate for each donor, and an average was used for statistical analysis) and compared their cytotoxicity against the CEM.NKR cells (which are naturally resistant to NK killing without HIV infection) after infecting the CEM.NKR cells with DH12 HIV virus (a dual-tropic virus) (FIG. 6C). Siglec-9⁺ CD56^{dim} NK cells exhibited higher cytotoxicity and were more degranulated (FIG. 5C) against HIV-infected cells compared to Siglec-9⁻ CD56^{dim} NK cells. These data are in contrast with a recent publication by Jandus et al. (Jandus et al., 2014, The Journal of clinical investigation 124, 1810-1820), where sorted Siglec-9⁺ CD56^{dim} NK cells exhibited lower activation against the K562 cancer cell line compared to Siglec-9⁻ CD56^{dim} NK cells. To examine if the cytotoxicity of the Siglec-9⁺ CD56^{dim} NK cells is target-dependent, the K562 cancer cell line was used as target cells and the sorted Siglec-9⁺ or Siglec-9⁻ CD56^{dim} NK cells from three healthy donors as effectors (FIG. 7). Indeed, results from assays using the K562 cancer cell line were in agreement with data from Jandus et al. (Jandus et al., 2014, The Journal of clinical investigation 124, 1810-1820) (FIG. 5C). These data suggest that the high cytotoxicity of Siglec-9⁺ CD56^{dim} NK cells is target-specific.

### Blocking Siglec-9 Enhances the Ability of NK Cells to Kill HIV-Infected Cells.

The preceding data suggest that the Siglec-9⁺ subset of NK cells may play a role in controlling HIV infection; however, the Siglec-9 molecule itself is an inhibitory receptor which can restrain the cytolytic ability of these otherwise cytotoxic Siglec-9⁺ NK cells. Indeed, it was found that blocking Siglec-9 (using an in-house Siglec-9 blocking antibody) enhanced the ability of donors' NK cells (from four donors) to kill HIV-infected cells (HUT78/SF2 cells) (FIG. 5D). These effects were not observed using Siglec-9^{depleted} NK cells, demonstrating that the antibody enhances NK cytotoxicity against HIV+ cells by specifically blocking Siglec-9 molecule. Similar results were obtained using CEM.NKR cells infected with DH12 HIV (FIG. 5E-F). Specifically, sorted Siglec-9⁺ CD56^{dim} NK cells showed higher cytotoxicity (FIG. 5E) and degranulation (FIG. 5F) in the presence of the Siglec-9 blocking antibody. These data support a model in which Siglec-9⁺ CD56^{dim} NK cells help to control HIV infection but are being restrained by the inhibitory nature of Siglec-9 receptor signaling.

### Generation of HIV Antibody-Sialidase Conjugates to Selectively Disrupt Siglec/Sialoglycan Interactions Between NK Cells and HIV-Infected Cells.

Many cells, not just HIV-infected ones, express sialoglycans, and Siglec-sialic acid interactions are important immune negative checkpoints against autoimmunity (Dharmadhikari et al., 2017, Sci Rep 7, 45319; Laubli et al., 2020, Cell Mol Life Sci 77, 593-605; Schwarz et al., 2015, Elife 4; Varki et al., 2012, Annals of the New York Academy of Sciences 1253, 16-36). Therefore, an approach that selectively targets HIV-infected cells is needed. Such a targeted approach has recently been developed in the cancer field (Xiao et al., 2016, Proc Natl Acad Sci USA 113, 10304-10309; Gray et al., 2020, Nat Chem Biol 16, 1376-1384). In that work, it was found that conjugating sialidase (the enzyme that removes Sialic acid from glycans) to trastuzumab (Herceptin; an antibody against HER2⁺ breast cancer cells) selectively desialylated HER2⁺ breast cancer cells. This trastuzumab-sialidase conjugate prevented Siglec/Sialic acid-binding (both Siglec-7 and Siglec-9) and enhanced anti-tumor NK activity against HER2⁺ but not HER2⁻ cells (Xiao et al., 2016, Proc Natl Acad Sci USA 113, 10304-10309). Importantly, in an in vivo mouse model of breast cancer, antibody-sialidase conjugates were safe, effective and exhibited the low off-target activity and the high chemical stability needed for in vivo use (Gray et al., 2020, Nat Chem Biol 16, 1376-1384).

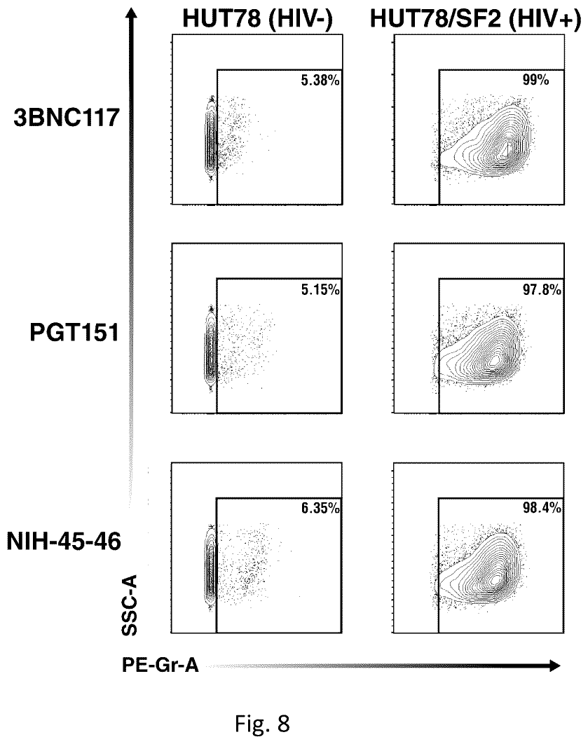

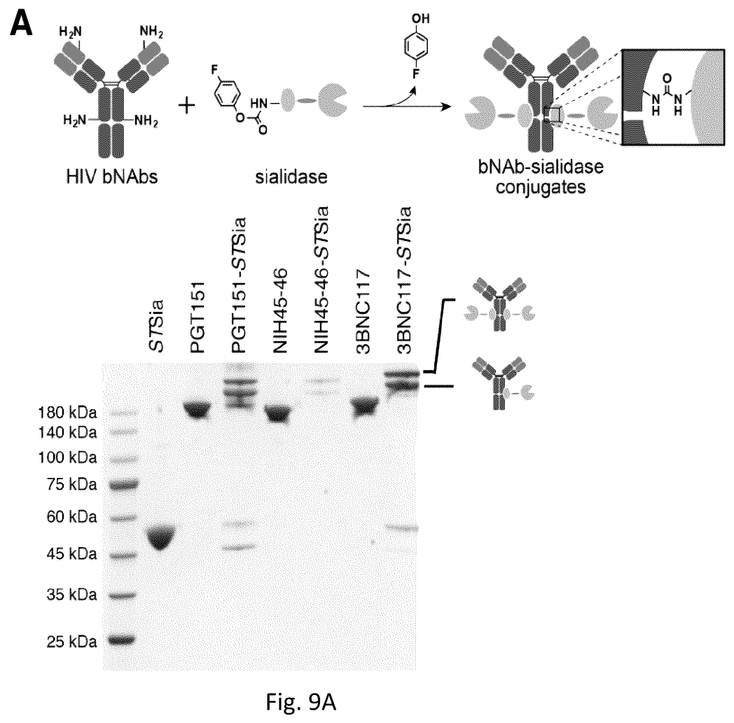

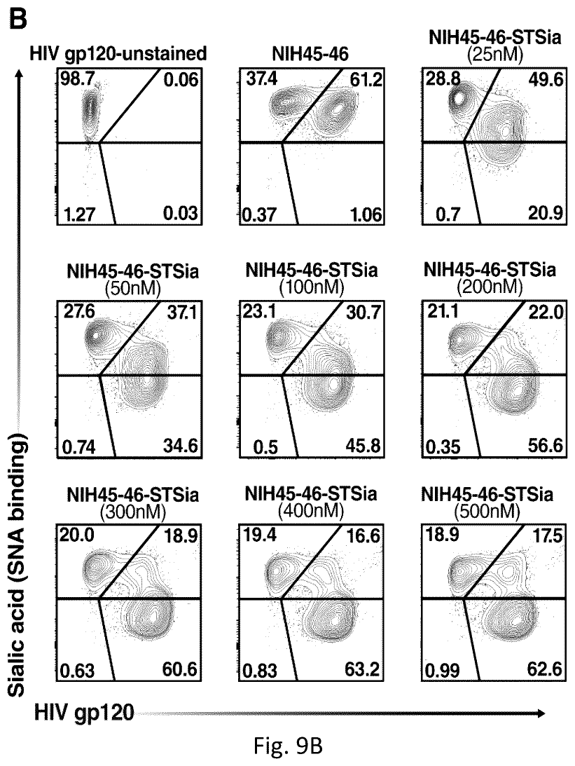

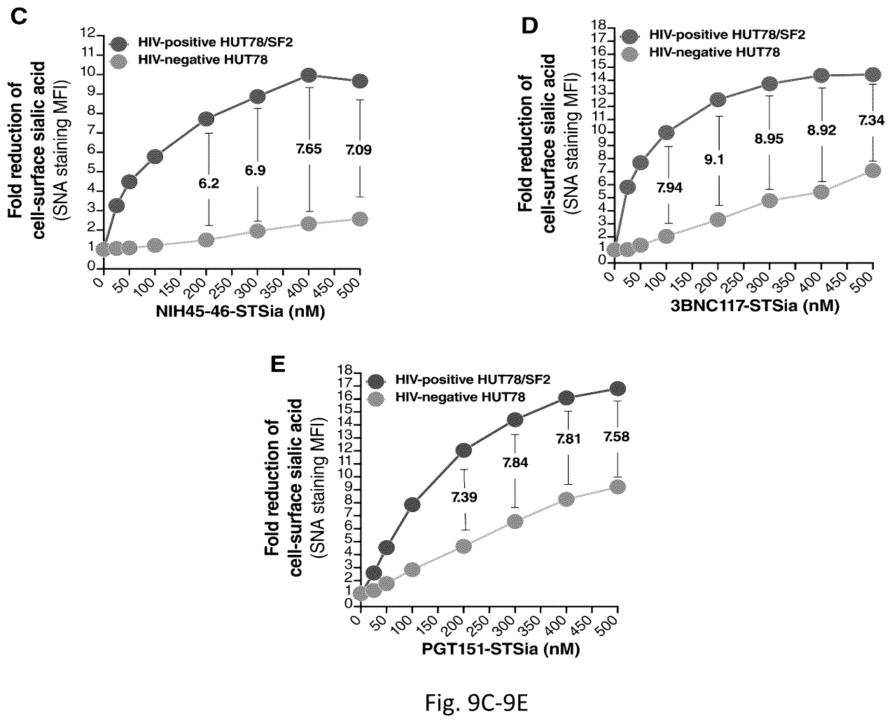

A similar approach was employed by conjugating Sialidase to HIV broadly neutralizing antibodies (bNAbs). First, DNA constructs of three HIV bNAbs (3BNC117, PGT151, and NIH45-46) were used to produce antibodies using Expi293F cells (Wise et al., 2020, J Clin Invest 130, 827-837). Purified antibodies highly bind to HIV⁺ cells in vitro (FIG. 8). The proximity-induced antibody labeling (pClick) technology (Yu et al., 2018, Bioconjugate chemistry 29, 3522-3526) was employed to conjugate these antibodies to Sialidase (from Salmonella typhimurium; STSia). pClick allows site-specific labeling of native antibodies with payloads under mild conditions, thus minimizing the disruption of antigen and Fc receptor binding. This approach was recently proven safe and effective in vivo (Gray et al., 2020, Nat Chem Biol 16, 1376-1384). To site-specifically conjugate the HIV bNAbs with Sialidase using pClick, first 4-fluorophenyl carbamate lysine was genetically incorporated into the Glu25 position of a FB fused with Sialidase using the genetic code expansion technology (FIG. 9A top). Next, bNAb-STSia conjugates were prepared by incubating bNAbs with 16 equivalents of FB-STSia for 48 hours. The conjugates were characterized by SDS-PAGE and determined an enzyme/antibody ratio of 1.0 (FIG. 9A bottom).

HIV bNAb-Sialidase Conjugates Selectively Desialylated HIV-Infected Cells.

Next, whether the HIV bNAb-sialidase conjugates can selectively remove sialic acid from HIV-infected cells was tested. A mixture of HUT78/SF2 (HIV⁺ cells) and HUT78 (HIV-negative cells) was treated with each of the three bNAb-sialidase conjugates at escalating doses. Cells were then stained with a secondary antibody for anti-HIV antibody and SNA (a lectin that binds specifically to Sialic acid). Treatment with the NIH45-56-STSia conjugate selectively desialylated HIV⁺ cells, while HIV-negative cells were minimally affected (FIG. 9B-C). Similar results were obtained with 3BNC117-STSia conjugate (FIG. 9D) and PGT151-STSia conjugate (FIG. 9E).

HIV bNAb-Sialidase Conjugates Enhance NK Cytotoxicity Against HIV-Infected Cells.

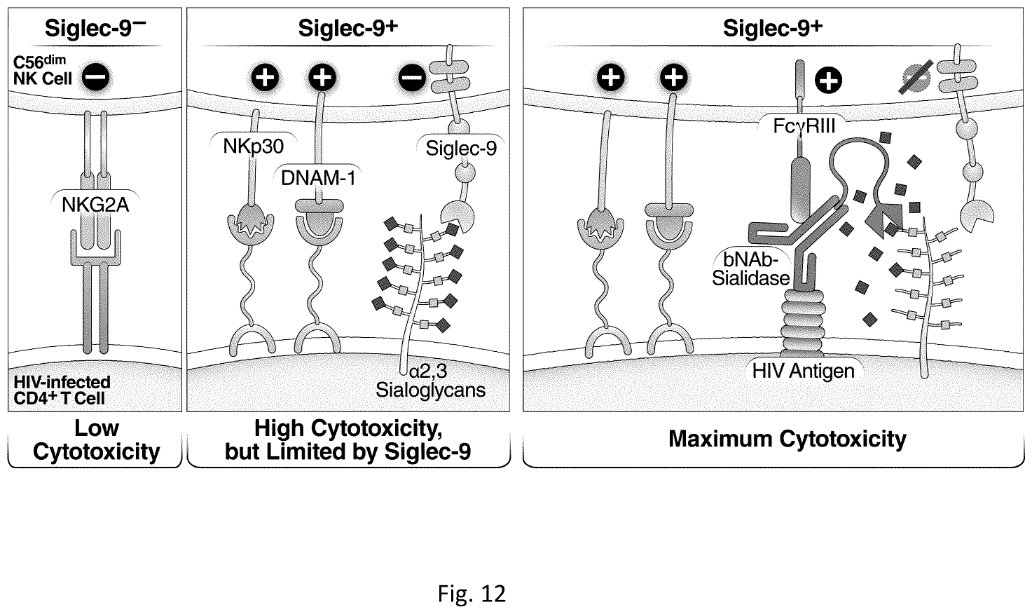

Next, whether these conjugates can enhance NK-mediated cytotoxicity against HIV-infected cells was tested. As targets, CEM.NKR CCR5⁺ Luc+ cells were utilized as these are 1) infectable with HIV (FIG. 10A); 2) naturally resistant to NK killing without HIV infection; and 3) express luciferase as a marker of HIV infection; and 4) can be desialylated by STSia to remove Siglec-9 ligands from their cell-surface (FIG. 10B). This desialylation of CEM.NKR cells enhances NK activity against them (FIG. 10C). The ability of each of the three conjugates to potentiate killing of HIV HXB2-infected cells by primary NK cells (from multiple HIV-uninfected donors) was tested. NK cells alone were able to reduce luciferase units (a marker of HIV-infected cells remaining in the culture) by 2.5 fold. The addition of 50 nM NIH45-46 enhanced this to 7.3 fold. The addition of 50 nM NIH45-46-sialidase conjugate enhanced the reduction by 42.4 fold (P=0.04; FIG. 11A; almost reduced to the same levels of uninfected cultures). Similar results were obtained using 200 nM of the antibody or antibody-sialidase conjugate (FIG. 11A). Similar results were also obtained using 3BNC117 or PGT151 conjugates (FIG. 11B-C). Cytotoxicity was examined by measuring levels of lactate dehydrogenase (LDH) released into the supernatant from damaged cells [normalized to the background; by luminescence] and similar results were obtained—the bNAbs-STSIA conjugates enhanced the ability of NK cells to kill HIV-infected cells compared to the unconjugated bNAbs (FIG. 11D-F). Together these data suggest that disrupting Siglec/sialoglycan interactions by selectively desialylating HIV-infected cells, using antibody-Sialidase conjugates, represents a novel proof-of-concept approach to enhance NK cell cytotoxicity against antigen-producing HIV-infected cells (FIG. 12).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A composition comprising a targeting domain specific for binding to a human immunodeficiency virus (HIV) antigen and a domain for desialylation of HIV-infected cells.

2. The composition of claim 1, wherein the domain for desialylation of HIV-infected cells comprises a neuraminidase enzyme, or a fragment or variant thereof.

3. The composition of claim 2, wherein the neuraminidase enzyme is selected from the group consisting of NEU1, NEU2, NEU3, NEU4, a bacterial neuraminidase, and a viral neuraminidase.

4. The composition of claim 1 comprising an anti-HIV antibody conjugated to a domain for desialylation of HIV-infected cells.

5. The composition of claim 4, wherein the anti-HIV antibody comprises PGDM1400, PGT121, or a combination thereof.

6. The composition of claim 4, wherein the domain for desialylation of HIV-infected cells comprises a neuraminidase enzyme, or a fragment or variant thereof.

7. The composition of claim 4, wherein the neuraminidase enzyme is selected from the group consisting of NEU1, NEU2, NEU3, NEU4, a bacterial neuraminidase, and a viral neuraminidase.

8. The composition of claim 1, further comprising a pharmaceutically acceptable excipient.

9. A method of preventing or treating HIV or a disease or disorder associated with HIV infection in a subject, the method comprising administering to the subject a composition of claim 1.

10. The method of claim 9, wherein the disease is acquired immunodeficiency syndrome (AIDS).

11. A nucleic acid molecule encoding a fusion molecule comprising targeting domain specific for binding to a human immunodeficiency virus (HIV) antigen and a domain for desialylation of HIV-infected cells.

12. The nucleic acid molecule of claim 11, wherein the domain for desialylation of HIV-infected cells comprises a neuraminidase enzyme, or a fragment or variant thereof.

13. The nucleic acid molecule of claim 11, wherein the neuraminidase enzyme is selected from the group consisting of NEU1, NEU2, NEU3, NEU4, a bacterial neuraminidase, and a viral neuraminidase.

14. The nucleic acid molecule of claim 11, wherein the nucleic acid molecule encodes an anti-HIV antibody conjugated to a domain for desialylation of HIV-infected cells.

15. The nucleic acid molecule of claim 11, wherein the nucleic acid molecule encodes PGDM1400 or PGT121.

16. A fusion molecule comprising targeting domain specific for binding to a human immunodeficiency virus (HIV) antigen and a domain for desialylation of HIV-infected cells.

17. The fusion molecule of claim 16, wherein the domain for desialylation of HIV-infected cells comprises a neuraminidase enzyme, or a fragment or variant thereof.

18. The fusion molecule of claim 16, wherein the neuraminidase enzyme is selected from the group consisting of NEU1, NEU2, NEU3, NEU4, a bacterial neuraminidase, and a viral neuraminidase.

19. The fusion molecule of claim 16, comprising an anti-HIV antibody conjugated to a domain for desialylation of HIV-infected cells.
